SEQUENCE LISTING (150 sequences)

SEQ ID NO: 1 - protein, 9 aa, SARS-CoV-2
VMVELVAEL

SEQ ID NO: 2 - protein, 9 aa, SARS-CoV-2
TLGVLVPHV

SEQ ID NO: 3 - protein, 9 aa, SARS-CoV-2
YLATALLTL

SEQ ID NO: 4 - protein, 9 aa, SARS-CoV-2
SLPGVFCGV

SEQ ID NO: 5 - protein, 9 aa, SARS-CoV-2
VLAWLYAAV

SEQ ID NO: 6 - protein, 9 aa, SARS-CoV-2
FLNRFTTTL

SEQ ID NO: 7 - protein, 9 aa, SARS-CoV-2
TLMNVLTLV

SEQ ID NO: 8 - protein, 9 aa, SARS-CoV-2
SMWALIISV

SEQ ID NO: 9 - protein, 9 aa, SARS-CoV-2
VLLSVLQQL

SEQ ID NO: 10 - protein, 9 aa, SARS-CoV-2
MLFTMLRKL

SEQ ID NO: 11 - protein, 9 aa, SARS-CoV-2
ALWEIQQVV

SEQ ID NO: 12 - protein, 9 aa, SARS-CoV-2
TMADLVYAL

SEQ ID NO: 13 - protein, 9 aa, SARS-CoV-2
IFVDGVPFV

SEQ ID NO: 14 - protein, 9 aa, SARS-CoV-2
YLQPRTFLL

SEQ ID NO: 15 - protein, 9 aa, SARS-CoV-2
KIADYNYKL

SEQ ID NO: 16 - protein, 9 aa, SARS-CoV-2
SIIAYTMSL

SEQ ID NO: 17 - protein, 9 aa, SARS-CoV-2
VLNDILSRL

SEQ ID NO: 18 - protein, 9 aa, SARS-CoV-2
RLDKVEAEV

SEQ ID NO: 19 - protein, 9 aa, SARS-CoV-2
FIAGLIAIV

SEQ ID NO: 20 - protein, 9 aa, SARS-CoV-2
KLLEQWNLV

SEQ ID NO: 21 - protein, 9 aa, SARS-CoV-2
FLFLTWICL

SEQ ID NO: 22 - protein, 9 aa, SARS-CoV-2
TLACFVLAA

SEQ ID NO: 23 - protein, 9 aa, SARS-CoV-2
GLMWLSYFI

SEQ ID NO: 24 - protein, 9 aa, SARS-CoV-2
LLLDRLNQL

SEQ ID NO: 25 - protein, 9 aa, SARS-CoV-2
GMSRIGMEV

SEQ ID NO: 26 - protein, 9 aa, SARS-CoV-2
FIDTKRGVY

SEQ ID NO: 27 - protein, 9 aa, SARS-CoV-2
YTERSEKSY

SEQ ID NO: 28 - protein, 9 aa, SARS-CoV-2
PTDNYITTY

SEQ ID NO: 29 - protein, 9 aa, SARS-CoV-2
VVDYGARFY

SEQ ID NO: 30 - protein, 9 aa, SARS-CoV-2
EIDPKLDNY

SEQ ID NO: 31 - protein, 9 aa, SARS-CoV-2
GTDLEGNFY

SEQ ID NO: 32 - protein, 9 aa, SARS-CoV-2
TCDGTTFTY

SEQ ID NO: 33 - protein, 9 aa, SARS-CoV-2
CTDDNALAY

SEQ ID NO: 34 - protein, 9 aa, SARS-CoV-2
KSDGTGTIY

SEQ ID NO: 35 - protein, 9 aa, SARS-CoV-2
ISDYDYYRY

SEQ ID NO: 36 - protein, 9 aa, SARS-CoV-2
VVDKYFDCY

SEQ ID NO: 37 - protein, 9 aa, SARS-CoV-2
DTDFVNEFY

SEQ ID NO: 38 - protein, 9 aa, SARS-CoV-2
QADVEWKFY

SEQ ID NO: 39 - protein, 9 aa, SARS-CoV-2
AMDEFIERY

SEQ ID NO: 40 - protein, 9 aa, SARS-CoV-2
CNDPFLGVY

SEQ ID NO: 41 - protein, 9 aa, SARS-CoV-2
WMESEFRVY

SEQ ID NO: 42 - protein, 9 aa, SARS-CoV-2
WTAGAAAYY

SEQ ID NO: 43 - protein, 9 aa, SARS-CoV-2
TSNQVAVLY

SEQ ID NO: 44 - protein, 9 aa, SARS-CoV-2
KTSVDCTMY

SEQ ID NO: 45 - protein, 9 aa, SARS-CoV-2
LTDEMIAQY

SEQ ID NO: 46 - protein, 9 aa, SARS-CoV-2
SSPDDQIGY

SEQ ID NO: 47 - protein, 9 aa, SARS-CoV-2
LSPRWYFYY

SEQ ID NO: 48 - protein, 9 aa, SARS-CoV-2
YANRNRFLY

SEQ ID NO: 49 - protein, 9 aa, SARS-CoV-2
ATSRTLSYY

SEQ ID NO: 50 - protein, 9 aa, SARS-CoV-2
AGDSGFAAY

SEQ ID NO: 51 - protein, 9 aa, SARS-CoV-2
SSDNIALLV

SEQ ID NO: 52 - protein, 9 aa, SARS-CoV-2
VEKGVLPQL

SEQ ID NO: 53 - protein, 9 aa, SARS-CoV-2
SEVGPEHSL

SEQ ID NO: 54 - protein, 9 aa, SARS-CoV-2
AEYHNESGL

SEQ ID NO: 55 - protein, 9 aa, SARS-CoV-2
NESGLKTIL

SEQ ID NO: 56 - protein, 9 aa, SARS-CoV-2
EETGLLMPL

SEQ ID NO: 57 - protein, 9 aa, SARS-CoV-2
GETLPTEVL

SEQ ID NO: 58 - protein, 9 aa, SARS-CoV-2
HEVLLAPLL

SEQ ID NO: 59 - protein, 9 aa, SARS-CoV-2
NETLVTMPL

SEQ ID NO: 60 - protein, 9 aa, SARS-CoV-2
GEVITFDNL

SEQ ID NO: 61 - protein, 9 aa, SARS-CoV-2
HEGKTFYVL

SEQ ID NO: 62 - protein, 9 aa, SARS-CoV-2
GEAANFCAL

SEQ ID NO: 63 - protein, 9 aa, SARS-CoV-2
TEVVGDIIL

SEQ ID NO: 64 - protein, 9 aa, SARS-CoV-2
TEEVGHTDL

SEQ ID NO: 65 - protein, 9 aa, SARS-CoV-2
AEWFLAYIL

SEQ ID NO: 66 - protein, 9 aa, SARS-CoV-2
AELAKNVSL

SEQ ID NO: 67 - protein, 9 aa, SARS-CoV-2
HEETIYNLL

SEQ ID NO: 68 - protein, 9 aa, SARS-CoV-2
AESHVDTDL

SEQ ID NO: 69 - protein, 9 aa, SARS-CoV-2
QEYADVFHL

SEQ ID NO: 70 - protein, 9 aa, SARS-CoV-2
REVLSDREL

SEQ ID NO: 71 - protein, 9 aa, SARS-CoV-2
TEHSWNADL

SEQ ID NO: 72 - protein, 9 aa, SARS-CoV-2
FEYVSQPFL

SEQ ID NO: 73 - protein, 9 aa, SARS-CoV-2
GEVFNATRF

SEQ ID NO: 74 - protein, 9 aa, SARS-CoV-2
FERDISTEI

SEQ ID NO: 75 - protein, 9 aa, SARS-CoV-2
AEVQIDRLI

SEQ ID NO: 76 - protein, 9 aa, SARS-CoV-2
AEIRASANL

SEQ ID NO: 77 - protein, 9 aa, SARS-CoV-2
DEDDSEPVL

SEQ ID NO: 78 - protein, 9 aa, SARS-CoV-2
LQLPQGTTL

SEQ ID NO: 79 - protein, 9 aa, SARS-CoV-2
MEVTPSGTW

SEQ ID NO: 80 - protein, 9 aa, SARS-CoV-2
GDAALALLL

SEQ ID NO: 81 - protein, 9 aa, SARS-CoV-2
SELVIGAVI

SEQ ID NO: 82 - protein, 165 aa, Pyrococcus furiosis
SERMLKALNDQLNRELYSAYLYFAMAAYFEDLGLEGFANWMKAQAEEEIGHALRFYNYIYDRNGRVELDEIPKPPKEWESPLKAFEAAYEHEKFISKSIYELAALAEEEKDYSTRAFLEWFINEQVEEEASVKKILDKLKFAKDSPQILFMLDKELSARAPKLPG

SEQ ID NO: 83 - protein, 173 aa, Helicobacter pylori
ESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 84 - protein, 160 aa, Mycobacterium smegmatis
SARRTESDIQGFHATPEFGGNLQKVLVDLIELSLQGKQAHWNVVGSNFRDLHLQLDELVDFAREGSDTIAERMRALDAVPDGRSDTVAATTTLPEFPAFERSTADVVDLITTRINATVDTIRRVHDAVDAEDPSTADLLHGLIDGLEKQAWLIRSENRKV

SEQ ID NO: 85 - protein, 154 aa, Aquifex aeolicus
MQIYEGKLTAEGLRFGIVASRFNHALVDRLVEGAIDAIVRHGGREEDITLVRVPGSWEIPVAAGELARKEDIDAVIAIGVLIRGATPHFDYIASEVSKGLAQLSLELRKPITFGVITADTLEQAIERAGTKHGNKGWEAALSAIEMANLFKSLR

SEQ ID NO: 86 - protein, 4 aa, Artificial sequence
  synthetic sequence
GGGS

SEQ ID NO: 87 - protein, 12 aa, Artificial sequence
  synthetic sequence
GGGSGGGSGGGS

SEQ ID NO: 88 - protein, 24 aa, Artificial sequence
  synthetic sequence
GGGSGGGSGGGSGGGSGGGSGGGS

SEQ ID NO: 89 - protein, 3 aa, Artificial sequence
  synthetic sequence
SSG

SEQ ID NO: 90 - protein, 9 aa, Artificial sequence
  synthetic sequence
SSGSSGSSG

SEQ ID NO: 91 - protein, 11 aa, Artificial sequence
  synthetic sequence
SGGPSGGPSGG

SEQ ID NO: 92 - protein, 14 aa, Artificial sequence
  synthetic sequence
SSGSSGSSGSPVGG

SEQ ID NO: 93 - protein, 28 aa, Artificial sequence
  synthetic sequence
GGSGGSSPVSTPPTPSPSTPPTPSPGGS

SEQ ID NO: 94 - protein, 44 aa, Artificial sequence
  synthetic sequence
GGSGGSSPVSTPPTPSPSTPPTPSPSTPPTPSPSTPPTPSPGGS

SEQ ID NO: 95 - protein, 15 aa, Artificial sequence
  synthetic sequence
GGSGSPGSPGSPGGS

SEQ ID NO: 96 - protein, 21 aa, Artificial sequence
  synthetic sequence
SSGEAAAKEAAAKEAAAKSSG

SEQ ID NO: 97 - protein, 20 aa, Artificial sequence
  synthetic sequence
SSGEAAAKALEAEAAAKSSG

SEQ ID NO: 98 - protein, 16 aa, Artificial sequence
  synthetic sequence
GSSPGSSPGSSPGGGS

SEQ ID NO: 99 - protein, 33 aa, Artificial sequence
  synthetic sequence
GAAPAAAPAKQEAAAPAPAAKAEAPAAAPAAKA

SEQ ID NO: 100 - protein, 18 aa, Artificial sequence
  synthetic sequence
SSGGGPQGGPQGGPQSSG

SEQ ID NO: 101 - protein, 9 aa, Artificial sequence
  synthetic sequence
GGSSPGGSS

SEQ ID NO: 102 - protein, 19 aa, Artificial sequence
  synthetic sequence
GGSSPGGSSPGGSSPGGSS

SEQ ID NO: 103 - protein, 73 aa, Artificial sequence
  synthetic sequence
KLSGGGGSGGGGSGGGGSAEAWYNLGNAYYKQGDYQKAIEYYQKALELDPNNLQRSAGGGGSGGGGSGGGGAS

SEQ ID NO: 104 - protein, 15 aa, Artificial sequence
  synthetic sequence
GLNDIFEAQKIEWHE

SEQ ID NO: 105 - protein, 13 aa, Homo sapians
CASSIRSSYEQYF

SEQ ID NO: 106 - protein, 13 aa, Artificial sequence
  synthetic sequence
CASSYSISYEQYF

SEQ ID NO: 107 - protein, 15 aa, Artificial sequence
  synthetic sequence
CASSPVTGGIYGYTF

SEQ ID NO: 108 - protein, 12 aa, Artificial sequence
  synthetic sequence
CASSHNSYEQYF

SEQ ID NO: 109 - protein, 14 aa, Artificial sequence
  synthetic sequence
CASSSTGLPYGYTF

SEQ ID NO: 110 - protein, 15 aa, Artificial sequence
  synthetic sequence
CAMSSGGTSYGKLTF

SEQ ID NO: 111 - protein, 11 aa, Artificial sequence
  synthetic sequence
CARNTGNGFYF

SEQ ID NO: 112 - protein, 14 aa, Artificial sequence
  synthetic sequence
CALTLQNRDDKIIF

SEQ ID NO: 113 - protein, 14 aa, Artificial sequence
  synthetic sequence
CAVSESPFGNEKLT

SEQ ID NO: 114 - protein, 9 aa, Azospirillium
WLDGVTPSL

SEQ ID NO: 115 - protein, 9 aa, HCMV
NLVPMVATV

SEQ ID NO: 116 - protein, 12 aa, Adenovirus
ATFTSYRSWYLA

SEQ ID NO: 117 - protein, 13 aa, Influenza
PKYVKQNTLKLAT

SEQ ID NO: 118 - protein, 14 aa, Artificial sequence
  synthetic sequence
CASKRGLEVTEAFF

SEQ ID NO: 119 - protein, 13 aa, Artificial sequence
  synthetic sequence
CAWIPRSSYEQYF

SEQ ID NO: 120 - protein, 13 aa, Artificial sequence
  synthetic sequence
CASSFHGSTPQHF

SEQ ID NO: 121 - protein, 15 aa, Artificial sequence
  synthetic sequence
CATSDFGQSGGELFF

SEQ ID NO: 122 - protein, 13 aa, Artificial sequence
  synthetic sequence
CASSIRSAYEQYF

SEQ ID NO: 123 - protein, 13 aa, Artificial sequence
  synthetic sequence
CASSTRSSGELFF

SEQ ID NO: 124 - protein, 17 aa, Artificial sequence
  synthetic sequence
CASSQDEGTSGGYEQYF

SEQ ID NO: 125 - protein, 15 aa, Artificial sequence
  synthetic sequence
CASSEGSGGVDEQYF

SEQ ID NO: 126 - protein, 17 aa, Artificial sequence
  synthetic sequence
CASSIGSSGGVYNEQFF

SEQ ID NO: 127 - protein, 17 aa, Artificial sequence
  synthetic sequence
CASSQVGTSGGYNEQFF

SEQ ID NO: 128 - protein, 18 aa, Artificial sequence
  synthetic sequence
CASSQVGTSGGISYEQYF

SEQ ID NO: 129 - protein, 16 aa, Artificial sequence
  synthetic sequence
CASSQAGTSGAYEQYF

SEQ ID NO: 130 - protein, 13 aa, Artificial sequence
  synthetic sequence
CASSFQGYTEAFF

SEQ ID NO: 131 - protein, 11 aa, Artificial sequence
  synthetic sequence
CASSSVTEAFF

SEQ ID NO: 132 - protein, 17 aa, Artificial sequence
  synthetic sequence
CASSPYYSGGAENEQFF

SEQ ID NO: 133 - protein, 16 aa, Artificial sequence
  synthetic sequence
CASSPSDGISGNTIYF

SEQ ID NO: 134 - protein, 15 aa, Artificial sequence
  synthetic sequence
CASSYQTGASYGYTF

SEQ ID NO: 135 - protein, 17 aa, Artificial sequence
  synthetic sequence
CSARPGFVGGYNSPLHF

SEQ ID NO: 136 - protein, 16 aa, Artificial sequence
  synthetic sequence
CASSQGELAGDGEQYF

SEQ ID NO: 137 - protein, 14 aa, Artificial sequence
  synthetic sequence
CASSFGQGVPEAFF

SEQ ID NO: 138 - protein, 215 aa, Artificial sequence
  synthetic sequence
MPMGSLQPLATLYLLGMLVASCLGGLNDIFEAQKIEWHESGGPSGGPSGGSERMLKALNDQLNRELYSAYLYFAMAAYFEDLGLEGFANWMKAQAEEEIGHALRFYNYIYDRNGRVELDEIPKPPKEWESPLKAFEAAYEHEKFISKSIYELAALAEEEKDYSTRAFLEWFINEQVEEEASVKKILDKLKFAKDSPQILFMLDKELSARAPKLPG

SEQ ID NO: 139 - protein, 223 aa, Artificial sequence
  synthetic sequence
MPMGSLQPLATLYLLGMLVASCLGGLNDIFEAQKIEWHESGGPSGGPSGGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 140 - protein, 210 aa, Artificial sequence
  synthetic sequence
MPMGSLQPLATLYLLGMLVASCLGGLNDIFEAQKIEWHESGGPSGGPSGGSARRTESDIQGFHATPEFGGNLQKVLVDLIELSLQGKQAHWNVVGSNFRDLHLQLDELVDFAREGSDTIAERMRALDAVPDGRSDTVAATTTLPEFPAFERSTADVVDLITTRINATVDTIRRVHDAVDAEDPSTADLLHGLIDGLEKQAWLIRSENRKV

SEQ ID NO: 141 - protein, 204 aa, Artificial sequence
  synthetic sequence
MPMGSLQPLATLYLLGMLVASCLGGLNDIFEAQKIEWHESGGPSGGPSGGMQIYEGKLTAEGLRFGIVASRFNHALVDRLVEGAIDAIVRHGGREEDITLVRVPGSWEIPVAAGELARKEDIDAVIAIGVLIRGATPHFDYIASEVSKGLAQLSLELRKPITFGVITADTLEQAIERAGTKHGNKGWEAALSAIEMANLFKSLR

SEQ ID NO: 142 - protein, 15 aa, Artificial sequence
  synthetic sequence
CASSESSGGVDEQYF

SEQ ID NO: 143 - protein, 15 aa, Artificial sequence
  synthetic sequence
CASSESSGGVNEQYF

SEQ ID NO: 144 - protein, 17 aa, Artificial sequence
  synthetic sequence
CASSQEGTSGGVNEQFF

SEQ ID NO: 145 - protein, 14 aa, Artificial sequence
  synthetic sequence
CASSFGQGAPEAFF

SEQ ID NO: 146 - protein, 14 aa, Artificial sequence
  synthetic sequence
CASSFGQGAYEQYF

SEQ ID NO: 147 - protein, 16 aa, Artificial sequence
  synthetic sequence
CASSFGQRITKNIQYF

SEQ ID NO: 148 - protein, 4 aa, Artificial sequence
  synethic sequence
SGGV

SEQ ID NO: 149 - protein, 4 aa, Artificial sequence
  synthetic sequence
SFGQ

SEQ ID NO: 150 - protein, 5 aa, Artificial sequence
  synthetic sequence
  misc_feature (3)..(3): Xaa can be any naturally occurring amino acid
LPXTG

(19) United States
(12) Patent Application Publication
(10) Pub. No.: US 2024/0076356 A1
(43) Pub. Date: Mar. 7, 2024

(54) DISPLAY OF PEPTIDE-MHC (PMHC) ON MULTIMERIC PROTEIN SCAFFOLDS AND USES THEREOF

(71) Applicant: The Board of Trustees of the Leland Stanford Junior University, stanford, CA (US)

(72) Inventors: Mark M. Davis, Atherton, CA (US); Venkata Vamsee Aditya Mallajosyula, Palo Alto, CA (US)

(21) Appl. No.: 18/268,105

(22) PCT Filed: Dec. 20, 2021
(86) PCT No.: PCT/US2021/064378

§ 371 Date: Jun. 16, 2023

Related U.S. Application Data
(60) Provisional application No. 63/127,760, filed on Dec. 18, 2020.

(51) Int. Cl. C07K 14/79; C07K 14/74; C12N 9/10; G01N 33/569

(52) U.S. Cl. CPC C07K 14/79; C07K 14/70539; C12N 9/1085; G01N 33/56972; C07K 2319/90; C12Y 205/01078

(57) ABSTRACT

An antigen-specific T cell binding agent is provided, where a multivalent ‘spheromer’ system utilizes a scaffold of a self-assembling polypeptide nanoparticle, for example using selfassembling ferritin polypeptides. The system is compatible with current pMHC reagents, including both MHC-I and MHC-II molecules, and streptavidin reagents that allow ease-of-use. The spheromer assembly pipeline provides a consistent reagent across multiple batches of synthesis with ease of production. The defined geometry of the scaffold allows precise site-directed conjugation of pMHC, leading to a homogenous reagent. The spheromer binds cognate TCRs with a significantly higher avidity than a tetrameric reagent.

### CROSS-REFERENCE TO RELATED APPLICATIONS

This application claims the benefit of U.S. provisional Patent Application Ser. No. 63/127,760, filed on Dec. 18, 2020, the contents of which are herein incorporated by reference in their entirety.

### STATEMENT OF GOVERNMENT SUPPORT

This invention was made with Government support under contract AI057229 awarded by the National Institutes of Health. The Government has certain rights in the invention.

### INCORPORATION BY REFERENCE OF SEQUENCE LISTING

A Sequence Listing is provided herewith as a Sequence Listing text, STAN-1817_SEQ_LIST_ST25 created on Jun. 15, 2023, and having a size of 41,072 bytes. The contents of the Sequence Listing text are incorporated herein by reference in their entirety.

### BACKGROUND

The majority of T cells in most mammals, including human beings, express the αβ T cell receptor (TCR) and recognize a particular peptide bound to a major histocompatibility complex molecule (pMHC) expressed on target cells. The weak equilibrium dissociation constant (K_{D}˜1-200 μM) between the TCR and monomeric pMHC results in a transient complex that impedes easy detection. The development of the pMHC-tetramers (“tetramer”) technology, wherein conjugation of four pMHC molecules to streptavidin (SAv) results in the increased avidity of TCR binding, laid the foundation to circumvent this problem. Since then, several studies have increased the number of pMHCs to improve these reagents' ability to detect T cells with marginal affinity, such as pMHC dextramers that use dextran polymers to increase the number of pMHC. However, the detection of low affinity TCRs still remains challenging, partly due to an increased background from non-specific staining using higher valency platforms, thus negatively impacting the signal-to-noise ratio.

The disclosure herein provides an improvement on this technology.

### SUMMARY

A flexible system for the multivalent display of peptide-MHC (pMHC) and other molecules is provided. The system is built on the scaffold comprising a self-assembling protein, where the self-assembling protein assembles into a nanoparticle of greater than 4 subunits, and may assemble into a nanoparticle from about 12 to about 60 subunits, e.g. 12 subunits, 24 subunits, 30 subunits, 60 subunits, etc. Self-assembling proteins of interest include, without limitation, ferritin and mini-ferritin proteins; lumazine synthase, and the like. The assembled nanoparticle provide a scaffold for the multivalent display of pMHC, herein termed a “spheromer”. In some embodiments a spheromer comprises from about 12 to 24 monomers of a self-assembling ferritin protein.

The spheromer platform provides ease of production, defined site-specific conjugation of pMHC molecules that significantly reduces inter-batch variation, and compatibility with currently available pMHC molecules and streptavidin reagents. The spheromer binds both MHC-I and MHC-II restricted T cells with excellent specificity for pMHC, and at a significantly higher avidity than a tetramer. Furthermore, this reagent provides a better signal-to-noise ratio and detects a more diverse antigen-specific TCR repertoire when compared to equivalent tetramers or dextramers.

In some embodiments the scaffold for the spheromer is a genetically modified ferritin polypeptide. In some embodiments the ferritin is a “maxi-ferritin”. Exemplified maxi-ferritins include, without limitation, Pyrococcus furiosus maxi-ferritin; and Helicobacter pylori maxi-ferritin. Maxi-ferritin self-assembles to form a 24-subunit nanoparticle with an external diameter of ˜120 Å. In some embodiments the ferritin is a “mini-ferritin”. Exemplified mini-ferritins include, without limitation, Mycobacterium smegmatis mini-ferritin. Mini-ferritin proteins self-assemble to form a 12-subunit nanoparticle measuring about 9 nm in diameter.

In some embodiments the scaffold for the spheromer is a genetically modified lumazine synthase polypeptide (EC 2.5.1.78). The enzyme from a number of other bacteria and archaea forms icosahedral capsids with triangulation number T=1, comprising 60 subunits, with an outer diameter of around 16 nm. Exemplified lumazine synthase proteins include, without limitation, Aquifex aeolicus lumazine synthase.

In some embodiments, the self-assembling polypeptide, e.g. ferritin, lumazine synthase, etc. is modified to comprise a biotinylation signal sequence, optionally at the N-terminus of each polypeptide subunit. The biotinylation signal sequence may be separated from the self-assembling polypeptide sequence by a flexible linker, e.g. as shown in FIG. 6. In some embodiments the linker is linker (SG₂P)₂SG₂.

To assemble the spheromer, the genetically modified self-assembling polypeptides are allowed to self-assemble, and are functionalized with biotin. Biotinylation at the signal sequence allows the self-assembling polypeptide to bind to pMHC monomers at high affinity. The biotinylated scaffold is bound through an avidin moiety, e.g. streptavidin, to peptide-MHC molecules, e.g. two peptide-MHC molecules. Typically the spheromer comprises from about 6, from about 12, to about 30 pMHC molecules, for example where the number of pMHC proteins is about half the number of subunits.

Benefits of a spheromer in comparison to pMHC tetramers include increased affinity. For example affinity may be increased by about 10-fold, about 20-fold, about 50-fold, or more relative to a tetramer. The signal from staining is higher, e.g. about 2-fold, about 5-fold, about 10-fold higher, resulting in a better signal-to-noise ratio compared to other pMHC-formulations. The increased avidity and specificity allows the detection of a greater range of low-affinity T cells.

In other embodiments, the self-assembling polypeptide is genetically engineered with a tag other than biotinylation for conjugation to pMHC proteins, which tags comprise, without limitation, sortase-tag, where the pMHCs are modified to include the -LPXTG sortase A tag while the scaffold is modified to include an oligo glycine (G)n, with n=3-7 at the terminus to allow protein-protein ligation when incubated with the Sortase A ligase; Spy-tag, where the SpyCatcher sequence is appended to the C-terminus of the pMHC, while the scaffold is modified to include the Spy-tag, and the modified proteins are irreversibly conjugated upon incubation.

In some embodiments, methods are provided for labeling and/or detecting an immune cell according to specificity of its antigen receptor, for example a T cell antigen receptor (TCR) or a B cell antigen receptor. In the methods of the invention, an immune cell is contacted with a spheromer MHC-peptide complex, where the antigen receptor binds to a spheromer having a cognate MHC-antigen complex. The immune cells may be T cells, e.g. disease-relevant T cells. In some embodiments the spheromers are used to induce of antigen-specific immunological tolerance to treat autoimmune conditions. In some embodiments the spheromers are used to modulate anti-tumor/anti-viral immunity by inducing antigen-specific responses. In some embodiments the spheromers are conjugated to a detectable moiety. In some embodiments the spheromers are conjugated to a functional moiety, including without limitation a T cell stimulatory molecule.

Also provided are spheromer MHC-peptide complexes and kits for binding to an immune cell according to specificity of its antigen receptor. The methods, modified self-assembling polypeptides, MHC-peptide complexes and kits find use in a variety of applications related to the detection, purification and activation of antigen-specific immune cells, including T cells, such as those T cells involved in tumors, infectious diseases and autoimmune diseases.

For example, using spheromers for direct ex vivo study of SARS-CoV-2 specific CD8⁺ T cells, it was shown that T cells predicted to cross-react with seasonal human coronaviruses are significantly enriched in COVID-19 patients with mild symptoms in comparison to individuals with severe disease. These robust T cells to conserved epitopes detected in SARS-CoV-2 unexposed individuals and in those with mild disease can provide a key determinant in a successful adaptive immune response. Following these T cells using spheromer technology allows tracking SARS-CoV-2 vaccine immunity in vaccinated individuals. In some embodiments a spheromer composition is provided, wherein the spheromers comprise one or more SARS-CoV-2 peptides, including without limitation, the peptide sequences set forth in Table 1, e.g. 1 or more of SEQ ID NO:1-81; and may comprise each of SEQ ID NO:1-81.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1A-FIG. 1J: Assembly and characterization of the “spheromer”. (FIG. 1A) Molecular surface representation of pMHC (PDB ID: 3TO2, α-chain in light blue, β2m in dark blue and peptide in yellow) and SAv (PDB ID: 2RTG, monomer in red and rest in coral). (FIG. 1B) Model of a semi-saturated SAv-pMHC₂ intermediate that has two unoccupied biotin binding sites. A single orientation is shown for simplicity. (FIG. 1C) A model of spheromer that is assembled by the conjugation of six semi-saturated SAv-pMHC₂ molecules onto a functionalized maxi-ferritin scaffold (PDB ID:2JD6, grey). UCSF Chimera was used for molecular graphics. (FIG. 1D) Streptavidin gel-shift assay to evaluate the functionalization of maxi-ferritin. The flexible tethers engineered at the N-terminus of each monomer have one biotin binding site. (FIG. 1E) The formation of semi-saturated SAv-pMHC₂ monitored by streptavidin gel-shift assay. The MHC α-chain is biotinylated and shifts upon binding streptavidin. Data is shown for an MHC-II molecule. (FIG. 1F) Quantification of SAv-pMHC₂ formation as a function of pMHC and SAv reactant concentrations. The mean±SD of the measurements from three experiments is shown. (FIG. 1G) Size-exclusion chromatogram of the spheromer and its components. (FIG. 1H) Representative electron micrographs of negatively stained maxi-ferritin and spheromer. The SAv-pMHC₂ conjugated on the surface of the functionalized scaffold are indicated by red arrows. Scale bars, 20 nm. Validation of SAv-pMHC₂ conjugation to the spheromer using (FIG. 1I) anti-MHC (FIG. 1J) anti-streptavidin antibodies by ELISA (mean±SD). The experiment was performed with each sample in triplicates and repeated at least twice.

FIG. 2A-FIG. 2I: Spheromer binds both MHC-I and MHC-II restricted T cells with high avidity and specificity. (FIG. 2A) List of evaluated pMHC-TCR pairs. The binding of (FIG. 2B) TCR1 and (FIG. 2C) TCR3 to different formulations of BHW58-A*02:01 and Protein III-DRB1*15:01 respectively was determined by biolayer interferometry. An overlay of binding traces over a concentration series of the indicated pMHC formulation from one representative experiment is shown. Each binding experiment was repeated at least thrice. The mean±SD of the binding constant has been graphed. (FIG. 2D) Representative flow cytometry plots showing the binding of the indicated BHW58-A*02:01 formulations with equivalent pMHC concentration to a T cell line expressing TCR1. The non-specific binding of the different formulations was measured using untransduced Jurkat cells and a cell line expressing an irrelevant TCR. CD3 was measured as proxy for TCR expression. (FIG. 2E) Quantification of BHW58-A*02:01 binding measured by flow cytometry (mean±SD). The experiment was performed with each sample processed in duplicates and repeated at least twice. (FIG. 2F) The signal to noise ratio (S/N) of TCR1 binding to distinct BHW58-A*02:01 multivalent formulations. Mean±SD of the measurements from two independent experiments has been plotted. (FIG. 2G) Representative flow cytometry plots showing the binding of the indicated Protein III-DRB1*15:01 formulations with equivalent pMHC concentration to a T cell line expressing TCR3. The non-specific binding to Jurkat cells and an irrelevant TCR was also measured. CD3 was measured as proxy for TCR expression. (FIG. 2H) Quantification of Protein III-DRB1*15:01 binding (mean±SD) measured by flow cytometry. (FIG. 2I) The signal to noise ratio (S/N) of TCR3 binding to distinct Protein III-DRB1*15:01 multivalent formulations (mean±SD).

FIG. 3A-FIG. 3L: Spheromer detects a higher frequency of antigen-specific T cells with a more diverse TCR repertoire. Representative flow cytometry plots of CD8⁺ T cells stained with influenza-M1 and HCMV-pp65 (FIG. 3A) Tetramers or (FIG. 3B) Spheromers. CD8⁺ T cells were enriched by negative selection of PBMCs isolated from HLA-A*02:01 individuals. Enumeration of epitope-specific (FIG. 3C) M1 and (FIG. 3D) pp65 CD8⁺ T cells detected in healthy individuals using either tetramer or spheromer. Data from each donor (n=7) is represented by a point. A two-tailed, matched-pairs Wilcoxon signed-rank test was performed to determine the significance levels. (FIG. 3E) Volcano plots showing the variance in TRBV usage of M1-A*02:01 specific CD8⁺ T cells detected using the spheromer and other pMHC multimers. The TRBV genes enriched significantly (p-value≤0.01, Fisher's exact test) with the spheromer are highlighted in purple. (FIG. 3F) The distribution of spheromer derived, influenza-M1 specific TCR motifs identified by GLIPH2 and representative examples from each category. (FIG. 3G) A representative GLIPH2 cluster with specificity for influenza-M1 and composed of TCR sequences identified exclusively using the spheromer. (FIG. 3H) Representative flow cytometry plots showing the activation of a T cell line (expressing a TCR with “G % SG” motif) stimulated with an irrelevant or cognate (influenza-M1) peptide. The activation was measured by CD69 expression. The significance level was determined by a two-tailed, paired t-test. (FIG. 3I) Volcano plots representing the variance in TRBV usage of pp65-A*02:01 specific CD8⁺ T cells detected with distinct pMHC multimers. The TRBV genes enriched significantly (p-value≤0.01, Fisher's exact test) with the spheromer are highlighted in plum. (FIG. 3J) The distribution of spheromer derived, HCMV-pp65 specific TCR motifs identified by GLIPH2 and representative examples from each category. (FIG. 3K) A representative GLIPH2 cluster with specificity for HCMV-pp65 and comprised of spheromer derived TCR sequences exclusively. (FIG. 3L) Representative flow cytometry plots showing the activation of a T cell line (expressing a TCR with “G % LAGD” motif) stimulated with an irrelevant or cognate (HCMV-pp65) peptide. The activation was measured by CD69 expression. A two-tailed, paired t-test was performed to determine significance.

FIG. 4A-FIG. 4I: COVID-19 patients with divergent clinical outcomes exhibit distinct SARS-CoV-2 epitope specific CD8⁺ T cell responses. (FIG. 4A) The sequence conservation of SARS-CoV-2 epitopes across seasonal hCoVs. The epitopes were selected based on their biochemical properties and binding to HLA-A*02:01. These peptides span multiple SARS-CoV-2 coding regions (ORF1ab, S, M and N) and display varying degrees of sequence similarity. The pair-wise conservation score between SARS-CoV-2 and any given hCoV is indicated by the size of the bubble. The color represents the average conservation score across all hCoVs. (FIG. 4B) The enumeration of SARS-CoV-2 epitope specific CD8⁺ T cells in unexposed, pre-pandemic PBMC samples collected between April 2018-February 2019. Data from each donor (n=5) is represented by a dot. (FIG. 4C) A positive correlation was observed between the average sequence similarity of SARS-CoV-2 epitopes with hCoVs and the baseline frequency of SARS-CoV-2 epitope specific CD8⁺ T cells in healthy, unexposed individuals. (FIG. 4D) Evaluation of CD8⁺ T cell clonal expansion using single-cell TCR sequencing. SARS-CoV-2 specific CD8⁺ T cells in unexposed individuals were identified using spheromer. Each individual dot represents a distinct TCR clone. (FIG. 4E) The distribution of SARS-CoV-2 specific TCR motifs shared between unexposed individuals and COVID-19 patients. Each TCR motif is colored according to the WHO clinical score (mean of all COVID-19 patient scores in the representative cluster). A lower WHO score indicates milder symptoms. (FIG. 4F) UMAPs showing the distribution of SARS-CoV-2 specific CD8+ T cells across the naïve and memory subsets defined based on the expression of CD45RA and CCR7 markers; naïve (CD45RA⁺CCR7⁺), central memory (CM, CD45RA⁻ CCR7⁺), effector memory (EM, CD45RA⁻CCR7⁻), and effector memory expressing CD45RA (TEMRA, CD45RA⁺CCR7⁻) in healthy, unexposed individuals. (FIG. 4G) Quantification of the distribution of SARS-CoV-2 specific CD8+ T cells across the naïve and memory subsets in healthy, unexposed individuals. T cells against SARS-CoV-2 epitopes that are well conserved across seasonal hCoVs exhibited a predominant memory phenotype. (FIG. 4H) The principal component analysis (PCA) plot shows that COVID-19 patients with disparate clinical outcomes (n; mild=13, severe=11) are characterized by distinct SARS-CoV-2 epitope specific CD8⁺ T cell responses. (FIG. 4I) The frequency of SARS-CoV-2 epitope specific CD8⁺ T cells that contribute significantly to the first principal component (PC1, 67.2%) of the analysis showing divergent COVID-19 clinical outcomes. The adjusted p-value is reported as determined by Dunn's test corrected for multiple comparisons.

FIG. 5A-FIG. 5C: Selection and characterization of scaffold candidates. (FIG. 5A) The distribution of higher-order (n>10) oligomeric assemblies across all non-redundant entries (N=145,181) in the protein data bank. A preference for specific oligomeric states (n=12, 24 and 60) was observed. (FIG. 5B) Size-exclusion profile of purified proteins selected for developing a scaffold for the multivalent display of pMHC. (FIG. 5C) Summary table listing the yield and homogeneity for the scaffold candidates.

FIG. 6: Optimization of molecular tethers on the maxi-ferritin scaffold for SAv mediated conjugation of pMHC molecules. (A) 19 (L1-L19) unique linkers that varied in length and rigidity were tested as molecular tethers at the N-terminus of each maxi-ferritin subunit. The linker L6 (highlighted in green) was chosen for further characterization based on protein yield, homogeneity and SAv loading onto the functionalized scaffold. (B) Sequence of optimized maxi-ferritin scaffold for multivalent pMHC display. Sequence features: CD5 signal peptide (blue), biotinylation signal (black). The stop codon is indicated by a hyphen (red).

FIG. 7: Titration of semi-saturated SAv-pMHC₂ with the functionalized scaffold. Size-exclusion chromatography was used to determine the number of SAv-pMHC₂ molecules conjugated to the maxi-ferritin scaffold upon saturation. As shown, free reactants are undetectable when they are incubated at a molar ratio of 1:6 (scaffold:SAv-pMHC₂). The scaffold is not saturated at any molar ratio of SAv-pMHC₂<6. Also, there is no further shift in the elution volume of the assembled spheromer when the molar ratio of SAv-pMHC₂ is >6 with the concomitant observation of free SAv-pMHC₂.

FIG. 8A-FIG. 8B: pMHC-TCR binding affinity measurements by biolayer interferometry. The binding of (FIG. 8A) TCR2 and (FIG. 8B) TCR4 to different formulations of pp65-A*02:01 and HA-DRB1*04:01 respectively are shown. An overlay of binding traces from one representative experiment is shown. Each binding experiment was repeated at least thrice. The mean±SD of the binding constant has been plotted.

FIG. 9A-FIG. 9F: Staining of T cell lines with pMHC multimers. (FIG. 9A) Representative flow cytometry plots showing the binding of the indicated pp65-A*02:01 multimers at an equivalent pMHC concentration to a T cell line expressing TCR2. The non-specific binding to untransduced Jurkat cells and a cell line expressing an irrelevant TCR was also measured. CD3 expression was measured as a proxy for TCR. (FIG. 9B) Quantification of pp65-A*02:01 binding measured by flow cytometry (mean±SD). The experiment was performed with each sample processed in duplicates and repeated at least twice. (FIG. 9C) The signal to noise ratio (S/N) of TCR2 binding to distinct pp65-A*02:01 multivalent formulations. Mean±SD of the measurements from two independent experiments has been plotted. (FIG. 9D) Representative flow cytometry plots showing the binding of the indicated HA-DRB1*04:01 multimers formulated with equivalent pMHC concentration to cells expressing the cognate TCR (TCR4). The non-specific binding to Jurkat cells and an irrelevant TCR was also measured. CD3 was used as a proxy for TCR expression. (FIG. 9E) Quantification of HA-DRB1*04:01 binding (mean±SD) measured by flow cytometry. (FIG. 9F) The signal to noise ratio (S/N) of TCR4 binding to distinct HA-DRB1*04:01 multivalent formulations (mean±SD).

FIG. 10: Gating strategy and representative flow cytometry dot plots comparing tetramer and spheromer staining on the same sample. CD8⁺ T cells enriched from PBMCs by negative selection was equally distributed and stained with tetramers or spheromers (M1-A*02:01 and pp65-A*02:01). Cells were also stained with the gag-A*02:01 pMHC-multimer as an irrelevant ‘negative’ control.

FIG. 11A-FIG. 11D: Validation of the unique antigen-specific TCR motifs identified using spheromer. (FIG. 11A) Representative GLIPH2 cluster with influenza-M1 specificity that is composed exclusively of spheromer derived TCR sequences. (FIG. 11B) Representative flow cytometry plots showing the activation of a T cell line (expressing a TCR with the “SGGV” motif) stimulated with an irrelevant or cognate (influenza-M1) peptide. The activation was measured by CD69 expression. The significance level was determined by a two-tailed, paired t-test. (FIG. 11C) GLIPH2 cluster with HCMV-pp65 specificity. The cluster was composed of TCR sequences identified exclusively using the spheromer. (FIG. 11D) Representative flow cytometry plots showing the activation of a T cell line (expressing a TCR with “SFGQ” motif) stimulated with an irrelevant or cognate (HCMV-pp65) peptide. The activation was measured by CD69 expression. The significance level was determined by a two-tailed, paired t-test.

FIG. 12: Experimental validation of the predicted SARS-CoV-2 peptide binding to HLA-A*02:01. The binding of test peptides was monitored by an MHC stabilization assay using the TAP-deficient T2 cell line expressing HLA-A*02:01. A productive peptide binding event leads to the stabilization of MHC molecules on the surface of T2 cells that was monitored by flow cytometry. Fold-change (mean±SD) in MFI (test peptide/negative control) has been graphed. The experiment was performed with duplicates and repeated twice. The well characterized HLA-A*02:01 binding peptides (influenza-M1 and HCMV-pp65) were included as positive controls. The sequences corresponding to SARS-CoV-2 peptides are listed in Table 1.

FIG. 13A-FIG. 13D: Combinatorial staining with spheromer pools to resolve multiple antigen specificities simultaneously was adapted from an approach described previously. (FIG. 13A) The relative pMHC monomer concentrations for each flourophore label (Ax647, eFluor 450, PE and PE/Cyanine7) to detect multiple epitope specificities simultaneously was experimentally determined using a mixture of untransduced Jurkat cells with four T cell lines that had specificities to influenza-M1, HCMV-pp65, Azospirillum-BHW58 and EBV-BMLF1. UMAP embedding of the cell mixture stained with optimized pMHC concentrations for each fluorophore tag shows the resolution of distinct antigen specificities with almost no overlap. (B-D) The gating strategy and representative flow cytometry dot plots to characterize antigen-specific CD8⁺ T cells using combinatorial spheromer staining. (FIG. 13B) CD8⁺ T cells were enriched from PBMCs by negative selection before spheromer staining. Phenotypic subsets of CD8⁺ T cells were defined based on the expression of CD45RA and CCR7 markers; naïve (TN, CD45RA⁺CCR7⁺), central memory (TCM, CD45RA⁻CCR7⁺), effector memory (TEM, CD45RA-CCR7⁻), and effector memory expressing CD45RA (TEMRA, CD45RA⁺CCR7⁻). (FIG. 13C) Table illustrating the assignment of unique flourophore barcodes for the simultaneous detection of seven unique antigen specificities (P1-P7), wherein the P7 combination is assigned to an irrelevant gag-A*02:01 specificity. (FIG. 13D) Representative flow cytometry dot plots showing the deconvolution of antigen specificities based on the unique fluorophore barcodes.

FIG. 14A-FIG. 14B: Enumeration of SARS-CoV-2 specific CD8⁺ T cells in COVID-19 patients. The frequency of antigen-specific CD8⁺ T cells in mild (n=13) and severe (n=11) COVID-19 patient PBMC samples was determined with spheromers using a combinatorial staining approach. Data from each donor is represented by a dot.

FIG. 15A-FIG. 15C. Characterization of scaffold candidates for multivalent display of pMHC to enhance binding avidity. (FIG. 15A) A list of oligomeric proteins that have been characterized for the display of pMHC. (FIG. 15B) Staining of a cognate T cell line (TCR1) with the indicated pMHC-multimer formulations. (FIG. 15C) Quantification of BHW58-A*02:01 binding to different pMHC-multimer formulations measured by flow cytometry (mean±SD). The experiment was performed with each sample processed in duplicates and repeated at least twice. The signal to noise ratio (S/N) of TCR1 binding to distinct BHW58-A*02:01 multivalent formulations. Mean±SD of the measurements from two independent experiments has been plotted.

FIG. 16A-FIG. 16B: Staining of antigen-specific B-cells using the cognate spheromers. PBMCs isolated from volunteers ˜3-4 weeks after (A) influenza or (B) SARS-CoV-2 vaccination were stained with (FIG. 16A) influenza hemagglutinin (HA) or (FIG. 16B) SARS-CoV-2 receptor binding domain (RBD) displayed on the spheromer scaffold. The Ig-isotype of the antigen-specific B cells are also shown.

### DETAILED DESCRIPTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As used herein, “suitable conditions” for carrying out a synthetic step are explicitly provided herein or may be discerned by reference to publications directed to methods used in synthetic organic chemistry. The reference books and treatise set forth above that detail the synthesis of reactants useful in the preparation of compounds of the present invention, will also provide suitable conditions for carrying out a synthetic step according to the present invention.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution. The term lower alkyl will be used herein as known in the art to refer to an alkyl, straight, branched or cyclic, of from about 1 to 6 carbons.

The compounds of the invention, or their pharmaceutically acceptable salts may contain one or more asymmetric centers and may thus give rise to enantiomers, diastereomers, geometric isomers, individual isomers and other stereoisomeric forms that may be defined, in terms of absolute stereochemistry, as (R)- or (S)- or, as (D)- or (L)- for amino acids. The present invention is meant to include all such possible isomers, as well as, their racemic and optically pure forms. Optically active (+) and (−), (R)- and (S)-, or (D)- and (L)-isomers may be prepared using chiral synthons or chiral reagents, or resolved using conventional techniques, such as reverse phase HPLC. When the compounds described herein contain olefinic double bonds or other centers of geometric asymmetry, and unless specified otherwise, it is intended that the compounds include both E and Z geometric isomers. Likewise, all tautomeric forms are also intended to be included.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “sequence identity,” as used herein in reference to polypeptide or DNA sequences, refers to the subunit sequence identity between two molecules. When a subunit position in both of the molecules is occupied by the same monomeric subunit (e.g., the same amino acid residue or nucleotide), then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as the GCS program package (Devereux et al., Nucleic Acids Res. 12:387, 1984), BLASTP, BLASTN, FASTA (Atschul et al., J. Molecular Biol. 215:403, 1990).

By “protein variant” or “variant protein” or “variant polypeptide” herein is meant a protein that differs from a wild-type protein by virtue of at least one amino acid modification. The parent polypeptide may be a naturally occurring or wild-type (WT) polypeptide, or may be a modified version of a WT polypeptide. Variant polypeptide may refer to the polypeptide itself, a composition comprising the polypeptide, or the amino sequence that encodes it. Preferably, the variant polypeptide has at least one amino acid modification compared to the parent polypeptide, e.g. from about one to about ten amino acid modifications, and preferably from about one to about five amino acid modifications compared to the parent.

By “parent polypeptide”, “parent protein”, “precursor polypeptide”, or “precursor protein” as used herein is meant an unmodified polypeptide that is subsequently modified to generate a variant. A parent polypeptide may be a wild-type (or native) polypeptide, or a variant or engineered version of a wild-type polypeptide. Parent polypeptide may refer to the polypeptide itself, compositions that comprise the parent polypeptide, or the amino acid sequence that encodes it.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. “Amino acid analogs” refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acid modifications disclosed herein may include amino acid substitutions, deletions and insertions, particularly amino acid substitutions. Variant proteins may also include conservative modifications and substitutions at other positions of the cytokine and/or receptor (e.g., positions other than those involved in the affinity engineering). Such conservative substitutions include those described by Dayhoff in The Atlas of Protein Sequence and Structure 5 (1978), and by Argos in EMBO J., 8:779-785 (1989). For example, amino acids belonging to one of the following groups represent conservative changes: Group I: Ala, Pro, Gly, Gln, Asn, Ser, Thr; Group II: Cys, Ser, Tyr, Thr; Group Ill: Val, lie, Leu, Met, Ala, Phe; Group IV: Lys, Arg, His; Group V: Phe, Tyr, Trp, His; and Group VI: Asp, Glu. Further, amino acid substitutions with a designated amino acid may be replaced with a conservative change.

The term “isolated” refers to a molecule that is substantially free of its natural environment. For instance, an isolated protein is substantially free of cellular material or other proteins from the cell or tissue source from which it is derived. The term refers to preparations where the isolated protein is sufficiently pure to be administered as a therapeutic composition, or at least 70% to 80% (w/w) pure, more preferably, at least 80%-90% (w/w) pure, even more preferably, 90-95% pure; and, most preferably, at least 95%, 96%, 97%, 98%, 99%, or 100% (w/w) pure. A “separated” compound refers to a compound that is removed from at least 90% of at least one component of a sample from which the compound was obtained. Any compound described herein can be provided as an isolated or separated compound.

The terms “subject,” “individual,” and “patient” are used interchangeably herein to refer to a mammal being assessed for treatment and/or being treated. In some embodiments, the mammal is a human. The terms “subject,” “individual,” and “patient” encompass, without limitation, individuals having a disease. Subjects may be human, but also include other mammals, particularly those mammals useful as laboratory models for human disease, e.g., mice, rats, etc.

The term “sample” with reference to a patient encompasses blood and other liquid samples of biological origin, solid tissue samples such as a biopsy specimen or tissue cultures or cells derived therefrom and the progeny thereof. The term also encompasses samples that have been manipulated in any way after their procurement, such as by treatment with reagents; washed; or enrichment for certain cell populations, such as diseased cells. The definition also includes samples that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's diseased cell, e.g., a sample comprising polynucleotides and/or polypeptides that is obtained from a patient's diseased cell (e.g., a cell lysate or other cell extract comprising polynucleotides and/or polypeptides); and a sample comprising diseased cells from a patient. A biological sample comprising a diseased cell from a patient can also include non-diseased cells.

The term “diagnosis” is used herein to refer to the identification of a molecular or pathological state, disease or condition in a subject, individual, or patient.

The term “prognosis” is used herein to refer to the prediction of the likelihood of death or disease progression, including recurrence, spread, and drug resistance, in a subject, individual, or patient. The term “prediction” is used herein to refer to the act of foretelling or estimating, based on observation, experience, or scientific reasoning, the likelihood of a subject, individual, or patient experiencing a particular event or clinical outcome. In one example, a physician may attempt to predict the likelihood that a patient will survive.

As used herein, the terms “treatment,” “treating,” and the like, refer to administering an agent, or carrying out a procedure, for the purposes of obtaining an effect on or in a subject, individual, or patient. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of cancer in a mammal, particularly in a human, and includes: (a) inhibiting the disease, i.e., arresting its development; and (b) relieving the disease or its symptoms, i.e., causing regression of the disease or its symptoms.

Treating may refer to any indicia of success in the treatment or amelioration or prevention of a disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician.

As used herein, a “therapeutically effective amount” refers to that amount of the therapeutic agent sufficient to treat or manage a disease or disorder. A therapeutically effective amount may refer to the amount of therapeutic agent sufficient to delay or minimize the onset of disease. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

“In combination with”, “combination therapy” and “combination products” refer, in certain embodiments, to the concurrent administration of agents described herein in combination with additional therapies or agents, e.g. surgery, radiation, chemotherapy, and the like. When administered in combination, each component can be administered at the same time or sequentially in any order at different points in time. Thus, each component can be administered separately but sufficiently closely in time so as to provide the desired effect.

“Concomitant administration” means administration of one or more components, such as engineered proteins and cells, known therapeutic agents, etc. at such time that the combination will have a therapeutic effect. Such concomitant administration may involve concurrent (i.e. at the same time), prior, or subsequent administration of components. A person of ordinary skill in the art would have no difficulty determining the appropriate timing, sequence and dosages of administration.

The use of the term “in combination” does not restrict the order in which agents are administered. A first agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second agent.

Self-assembling polypeptides. Self-assembling polypeptides, as used herein, refers to polypeptides that assemble into higher order nanoparticle structures, particularly nanoparticles of greater than 4 subunits, usually nanoparticles of greater than 12 subunits.

In some embodiments a self-assembling polypeptide is a ferritin. Ferritins are a family of protein cages that play a key role in iron sequestration and are highly evolutionary ubiquitous. Ferritins from diverse organisms including animals, plants and bacteria have been isolated and crystallized. Although the DNA and amino acid sequences for the ferritins vary, their well conserved three dimensional tertiary structures indicate identical or similar monomer folding.

The ferritin superfamily can be broken into three sub-families: the classical ferritins (Ftn), the bacterioferritins (Bfr), and the DNA-binding proteins from starved cells (Dps). The Ftn and Bfr proteins are considered maxi-ferritins, whereas Dps proteins are mini-ferritins. These three sub-families share the same characteristic four-helix bundle fold. The Ftn proteins are found in all three domains of life (eukarya, archaea and bacteria) and are typical members of ferritin family. The Bfr proteins have identical quaternary structure to the Ftn proteins; however they are restricted to bacteria and archaea. The Dps proteins form a smaller molecule with a lower iron storage capacity than the Ftn and Bfr proteins and utilize unique ferroxidase sites.

Maxi-ferritins, for example Pyrococcus furiosus maxi-ferritin; and Helicobacter pylori maxi-ferritin, self-assemble to form a 24-subunit nanoparticle with an external diameter of ˜120 Å. In some embodiments a maxi-ferritin sequence is Pyrococcus furiosis, SEQ ID NO:82, SERMLKALNDQLNRELYSAYLYFAMAAYFEDLGLEGFANWMKAQAEEEIGHALRFYNYIYDRNGRVELD EIPKPPKEWESPLKAFEAAYEHEKFISKSIYELAALAEEEKDYSTRAFLEWFINEQVEEEASVKKILDKLKF AKDSPQILFMLDKELSARAPKLPG.

Helicobacter pylori maxi-ferritin scaffold has the amino acid sequence, SEQ ID NO:83 ESQVRQQFSKDIEKLLNEQVNKEMQSSNLYMSMSSWCYTHSLDGAGLFLFDHAAEEYEHAKKLIIFLNE NNVPVQLTSISAPEHKFEGLTQIFQKAYEHEQHISESINNIVDHAIKSKDHATFNFLQWYVAEQHEEEVLFK DILDKIELIGNENHGLYLADQYVKGIAKSRKS.

Mini-ferritin Dps proteins form cage-like oligomers similar to the maxi-ferritins but made up of only twelve monomers. The crystal structure of dodecameric E. coli Dps reveals a hollow protein with 32 (tetrahedral) point group symmetry. The Dps dodecamer measures ˜9 nm in diameter and has a central cavity of ˜4.5 nm which can hold an iron core of up to ˜500 Fe3+ iron ions. Similarly to the maxi-ferritins, the Dps monomer folds into a four-helix bundle (the A, B, C and D helices).

In some embodiments a mini-ferritin is Mycobacterium smegmatis mini-ferritin, which sequence can be accessed at Genbank A0R647, SEQ ID NO:84 SARRTESDIQGFHATPEFGGNLQKVLVDLIELSLQGKQAHWNVVGSNFRDLHLQLDELVDFAREGSDTIA ERMRALDAVPDGRSDTVAATTTLPEFPAFERSTADVVDLITTRINATVDTIRRVHDAVDAEDPSTADLLHG LIDGLEKQAWLIRSENRKV.

As an alternative to ferritins, a self-assembling lumazine synthase (LS, EC 2.5.1.78) polypeptide may be used. Lumazine synthase catalyzes the penultimate step in the biosynthesis of riboflavin. The enzyme from bacteria including, for example, Bacillus subtilis, Aquifex aeolicus, Bacillus anthracis, are exemplary. These proteins form icosahedral capsids with triangulation number T=1. The capsids have an outer diameter of around 16 nm and are built up by 12 pentameric units, thus consisting in total of 60 identical subunits, which are related by twofold-, threefold- and fivefold symmetry axes. The molecular weight of the icosahedral complex is around 960,000 Daltons. The quaternary assembly modes of the LS capsid structures are similar to those of the capsids of small icosahedral viruses. In some embodiments a lumazine synthase is Aquifex aeolicus LS, SEQ ID NO:85 MQIYEGKLTAEGLRFGIVASRFNHALVDRLVEGAIDAIVRHGGREEDITLVRVPGSWEIPVAAGELARKED IDAVIAIGVLIRGATPHFDYIASEVSKGLAQLSLELRKPITFGVITADTLEQAIERAGTKHGNKGWEAALSAIE MANLFKSLR.

In some embodiments, the self-assembling polypeptide, e.g. ferritin, lumazine synthase, etc., is modified to comprise a biotinylation signal sequence, optionally at the N-terminus of each polypeptide subunit. The biotinylation signal sequence may be separated from the self-assembling polypeptide sequence by a flexible linker, e.g. a linker selected from: SEQ ID NO:86 G₃S; SEQ ID NO:87 (G₃S)₃; SEQ ID NO:88 (G₃S)₆; SEQ ID NO:89 S₂G; SEQ ID NO:90 (S₂G)₃; SEQ ID NO:91 (SG₂P)₂SG₂; SEQ ID NO:92 (S₂G)₃SPVG₂; SEQ ID NO:93 (G₂S)₂SPV(STP₂TPSP)₂G₂S; SEQ ID NO:94 (G₂S)₂SPV(STP₂TPSP)₄G2S; SEQ ID NO:95 G₂S(GSP)₃G₂S; SEQ ID NO:96 S₂G(EA₃K)₃S₂G; SEQ ID NO:97 S₂GEA₃KALEAEA₃KS₂G; SEQ ID NO:98 (GS₂P)₃G₃S; SEQ ID NO:99 GA₂PA₃PAKQEA₃PAPA₂KAEAPA₃PA₂KA; SEQ ID NO:100 S₂G(G₂PQ)₃S₂G; SEQ ID NO:101 G₂S₂PG₂S₂; SEQ ID NO:142 G₂S₂PG₂S₂PG₂S₂PG₂S₂; and SEQ ID NO:102 KLSG4SG4SG4SAEAWYNLGNAY2KQGDYQ KAIEY2QKALELDPN2LQRSAG4SG4SG4AS. In some embodiments the linker is SEQ ID NO:91 (SG₂P)₂SG₂. In some embodiments the biotinylation signal is SEQ ID NO:103, GLNDIFEAQKIEWHE. A signal peptide may be included for secretion of the polypeptide, as known in the art.

Expression construct: The coding sequences of a self-assembling polypeptide, which may be modified to comprise a biotinylation signal sequence and linker, may be introduced on an expression vector into a cell for expression. The nucleic acid encoding the polypeptide is inserted into a vector for expression and/or integration. Many such vectors are available. The vector components generally include, but are not limited to, one or more of the following: an origin of replication, one or more marker genes, an enhancer element, a promoter, and a transcription termination sequence. Vectors include viral vectors, plasmid vectors, integrating vectors, and the like.

Expression vectors may contain a selection gene, also termed a selectable marker. This gene encodes a protein necessary for the survival or growth of transformed host cells grown in a selective culture medium or a truncated gene encoding a surface marker that allows for antibody based detection. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, or (d) enable surface antibody based detection for isolation via fluoresences activating cell sorting (FACS) or magnetic separation e.g. truncated forms of NGFR, EGFR, CD19.

Nucleic acids are “operably linked” when placed into a functional relationship with another nucleic acid sequence. For example, DNA for a signal sequence is operably linked to DNA for a polypeptide if it is expressed as a preprotein that signals the secretion of the polypeptide; a promoter or enhancer is operably linked to a coding sequence if it affects the transcription of the sequence; and a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous.

Expression vectors will contain a promoter that is recognized by the host organism and is operably linked to the ABD construct coding sequence. Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

Transcription from vectors in mammalian host cells may be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus LTR (such as murine stem cell virus), hepatitis-B virus and Simian Virus 40 (SV40), from heterologous mammalian promoters, e.g., the actin promoter, PGK (phosphoglycerate kinase), or an immunoglobulin promoter, or from heat-shock promoters, provided such promoters are compatible with the host cell systems. The early and late promoters of the SV40 virus are conveniently obtained as an SV40 restriction fragment that also contains the SV40 viral origin of replication.

Transcription by higher eukaryotes may be increased by inserting an enhancer sequence into the vector. Enhancers are cis-acting elements of DNA, usually about from 10 to 300 bp in length, which act on a promoter to increase its transcription. Enhancers are relatively orientation and position independent, having been found 5′ and 3′ to the transcription unit, within an intron, as well as within the coding sequence itself. Many enhancer sequences are now known from mammalian genes (globin, elastase, albumin, α-fetoprotein, and insulin). Typically, however, one will use an enhancer from a eukaryotic virus. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence, but is preferably located at a site 5′ from the promoter.

Host cells can be transfected with the above-described expression vectors for construct expression. Cells may be cultured in conventional nutrient media modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium ((MEM), Sigma), RPMI 1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression, and will be apparent to the ordinarily skilled artisan.

Methods for isolation of the recombinantly produced proteins can be practiced as known in the art.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “sequence identity,” as used herein in reference to polypeptide or DNA sequences, refers to the subunit sequence identity between two molecules. When a subunit position in both of the molecules is occupied by the same monomeric subunit (e.g., the same amino acid residue or nucleotide), then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as the GCS program package (Devereux et al., Nucleic Acids Res. 12:387, 1984), BLASTP, BLASTN, FASTA (Atschul et al., J. Molecular Biol. 215:403, 1990).

By “protein variant” or “variant protein” or “variant polypeptide” herein is meant a protein that differs from a wild-type protein by virtue of at least one amino acid modification. The parent polypeptide may be a naturally occurring or wild-type (WT) polypeptide, or may be a modified version of a WT polypeptide. Variant polypeptide may refer to the polypeptide itself, a composition comprising the polypeptide, or the amino sequence that encodes it. Preferably, the variant polypeptide has at least one amino acid modification compared to the parent polypeptide, e.g. from about one to about ten amino acid modifications, and preferably from about one to about five amino acid modifications compared to the parent.

By “parent polypeptide”, “parent protein”, “precursor polypeptide”, or “precursor protein” as used herein is meant an unmodified polypeptide that is subsequently modified to generate a variant. A parent polypeptide may be a wild-type (or native) polypeptide, or a variant or engineered version of a wild-type polypeptide. Parent polypeptide may refer to the polypeptide itself, compositions that comprise the parent polypeptide, or the amino acid sequence that encodes it.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. “Amino acid analogs” refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acid modifications disclosed herein may include amino acid substitutions, deletions and insertions, particularly amino acid substitutions. Variant proteins may also include conservative modifications and substitutions at other positions of the cytokine and/or receptor (e.g., positions other than those involved in the affinity engineering). Such conservative substitutions include those described by Dayhoff in The Atlas of Protein Sequence and Structure 5 (1978), and by Argos in EMBO J., 8:779-785 (1989). For example, amino acids belonging to one of the following groups represent conservative changes: Group I: Ala, Pro, Gly, Gln, Asn, Ser, Thr; Group II: Cys, Ser, Tyr, Thr; Group Ill: Val, lie, Leu, Met, Ala, Phe; Group IV: Lys, Arg, His; Group V: Phe, Tyr, Trp, His; and Group VI: Asp, Glu. Further, amino acid substitutions with a designated amino acid may be replaced with a conservative change.

The term “isolated” refers to a molecule that is substantially free of its natural environment. For instance, an isolated protein is substantially free of cellular material or other proteins from the cell or tissue source from which it is derived. The term refers to preparations where the isolated protein is sufficiently pure to be administered as a therapeutic composition, or at least 70% to 80% (w/w) pure, more preferably, at least 80%-90% (w/w) pure, even more preferably, 90-95% pure; and, most preferably, at least 95%, 96%, 97%, 98%, 99%, or 100% (w/w) pure. A “separated” compound refers to a compound that is removed from at least 90% of at least one component of a sample from which the compound was obtained. Any compound described herein can be provided as an isolated or separated compound.

### Compositions and Methods

As summarized above, methods for labeling and/or detecting a cell according to specificity of its antigen receptor (TCR) are provided. Also provided are spheromer MHC-peptide complexes and kits for binding to an immune cell according to specificity of its antigen receptor. The methods and spheromer MHC-peptide complexes find use in a variety of applications related to the detection and purification of antigen-specific cells, including without limitation those T cells involved in tumors, infectious diseases and autoimmune diseases.

Spheromer MHC-Peptide Complex. The spheromer MHC-peptide complex is prepared with major histocompatibility complex (MHC) protein subunits having a peptide bound in the antigen presentation site, bound to a self-assembling polypeptide scaffold. In some embodiments the self-assembling polypeptide is a ferritin polypeptide, including a ferritin polypeptide modified to comprise a biotinylation signal sequence. The spheromer assembly is a two-step process: i) Generation of a semi-saturated pMHC complex, and ii) Conjugation of the pMHC to the functionalized scaffold. In some embodiments the pMHC complex is functionalized with avidin, streptavidin, neutravidin, etc.; and the self-assembling polypeptide, e.g. a ferritin polypeptide, is biotinylated. The spheromer assembly is generated by incubating functionalized pMHC with the functionalized scaffold. Optionally the spheromer is further purified, e.g. using a size-exclusion column, etc.

The resulting spheromer MHC-peptide complex specifically bind to a cognate TCR or surface antibody (sAb) at high affinity, thereby allowing for the labeling, identification and separation of cells based on the specificity of the antigen receptor.

The spheromers can be adapted to label multiple cells simultaneously by utilizing a mixture of peptides that include different antigens; and/or a mixture of MHC proteins. In some embodiments, individual peptides or MHC proteins are labeled so as to be distinguished from other peptides or MHC complexes.

In other embodiments the spheromers can be adapted to label T cells and B cells simultaneously, e.g. to enhance an immune response. In such embodiments, a T cell antigen, i.e. a pMHC complex; and a B cell antigen, e.g. a polypeptide, polysaccharide, etc. that is not complexed to MHC, are both presented on a single spheromer. Such a spheromer can be generated, for example, by linking the scaffold to a mixture of B cell and T cell antigens. The peptide complexed with MHC (T cell antigen) and a non-complexed polypeptide (B cell antigen) may correspond to the same protein or immunogen. For example, the peptide complexed with MHC may be a fragment of the non-complexed polypeptide. The administration of such a B cell/T cell spheromer can enhance an immune response, e.g. during vaccination, by providing proximity of a responding B cell and T cell.

The MHC-peptide complex may be described by formula (I): α-β-P, where a is a soluble form of an α-chain of a class I or class II MHC protein; β is a soluble form of (i) a β-chain of a class II MHC protein, or (ii) β 2 microglobulin for a class I MHC protein; and P is a peptide comprising a TCR-recognition region; and where P is bound in the groove formed by two membrane distal domains of (i) the α-chain for a class I MHC protein or (ii) α-chain and the β-chains for a class II MHC protein.

Peptides. The peptide may comprise any T cell epitope or minimal antigenic determinant that provides for specific binding of the MHC-peptide complex to the TCR. Any T cell epitope of interest, or at least minimal antigenic determinant versions of the same, can be utilized in the subject complexes.

Generally the TCR-recognition region is a linear amino acid sequence of 5 or more residues, such as 6 or more, 8 or more, 10 or more, 12 or more, 13 or more, 14 or more, 16 or more, or 18 or more residues, e.g., 5, 6, 7, 8, 9, 10, 11, 12, 13, 14, 15, 16, 17, or 18, or even more amino acid residues. Usually the TCR-recognition region is between 5 and 30 amino acid residues, such as between 5 and 20 residues, between 6 and 18 amino acid residues, or between 8 and 17 residues, e.g., between 8 and 12 amino acid residues, or between 13 and 17 amino acid residues.

In some embodiments, the MHC-peptide complex comprises an MHC class I molecule, for example human HLA A, B or C; β 2 microglobulin, and a peptide with a core TCR-recognition region of between 6 and 12 amino acid residues, such as between 8 and 12 residues.

In other embodiments, the MHC-peptide complex comprises an alpha chain and a beta chain of an MHC class II molecule, for example human HLA DP, DM, DQ, DR, DOA/B; and a peptide with a core TCR-recognition region of between 6 and 20 amino acid residues, such as between 10 and 18 residues or between 13 and 17 amino acid residues.

The peptides may have a sequence derived from a wide variety of proteins. Many T cell epitopes are known in the art, and include a variety of autoantigens, tumor antigens, allergens, pathogen antigens, and the like, as known in the art. Any convenient epitopic sequences from a number of antigens may be utilized. Alternatively, the epitopic sequence may be empirically determined, by isolating and sequencing peptides bound to native MHC proteins, by synthesis of a series of peptides from the target sequence, then assaying for T cell reactivity to the different peptides, or by producing a series of binding complexes with different peptides and quantitating the T cell binding. For example, see De Groot et al. (2010) Methods in Microbiology 37:35-66, “Use of Bioinformatics to Predict MHC Ligands and T-Cell Epitopes: application to Epitope-Driven Vaccine Design”, herein specifically incorporated by reference. Preparation of fragments, identifying sequences, and identifying the minimal sequence is amply described in U.S. Pat. No. 5,019,384, iss. May 28, 1991, and references cited therein. These peptide sequences may be utilized in the T-cell recognition regions of the subject peptides.

The subject peptides may be prepared in a variety of ways. Conveniently, they can be synthesized using conventional techniques employing automatic synthesizers, or may be synthesized manually. Alternatively, DNA sequences can be prepared that encode the peptide of interest, which are cloned and expressed to provide the desired peptide. Peptide fragments may be produced by recombinant methods, for example as a fusion to a polypeptide with a tag for purification, allowing purification of the fusion protein by means of affinity reagents, followed by proteolytic cleavage, usually at an engineered site to yield the desired peptide fragment (see for example Driscoll et al. (1993) J. Mol. Bio. 232:342-350). The peptides may also be purified using any convenient techniques, including, for example, chromatography on ion exchange materials, separation by size, immunoaffinity chromatography and electrophoresis.

Detectable Moieties. As used herein, the term “detectable moiety” refers a moiety that can be detected by a variety of methods including fluorescence, electrical conductivity, radioactivity, size, and the like. The detectable moiety may be of a chemical (e.g., carbohydrate, lipid, etc.), peptide or nucleic acid nature although it is not so limited. The detectable moiety may be directly or indirectly detectable. The detectable moiety can be detected directly for example by its ability to emit and/or absorb light of a particular wavelength. A detectable moiety can be detected indirectly by its ability to bind, recruit and, in some cases, cleave (or be cleaved by) another compound, thereby emitting or absorbing energy. An example of indirect detection is the use of an enzyme label that cleaves a substrate into visible products.

Any convenient detectable moiety may be utilized in the subject peptides or complexes. Detectable moieties of interest include, but are not limited to, fluorophores, dyes, enzymes or enzyme substrates, chemiluminescers, specific binding moieties or their partners, particles, radioisotopes, affinity tags or other directly or indirectly detectable agent. In certain cases, the detectable moiety has a light detectable characteristic, e.g., a fluorophore, such as fluorescein isothiocyanate (FITC), Texas Red, Cy3, Cy5, phycoerythrin, allophycocyanin, 5,6-carboxymethyl fluorescein, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7.

The detectable moieties may also be specific binding moieties such as receptors, antibodies or antibody fragments or their corresponding antigen, epitope or hapten, or ligand binding partners. Detection of such moieties is accomplished by any convenient techniques. In certain embodiments, the detectable moiety is biotin. In some instances, the detectable moiety is an affinity tag that is suitable for use in methods of purification of T cells, e.g., by binding to a chromatography column. The detectable moiety may be utilized in the separation and/or purification of labeled T cell from unlabeled T cells.

MHC Molecule. The complex is prepared with major histocompatibility complex protein subunits having a homogeneous population of peptides bound in the antigen presentation site.

The MHC proteins may be from any mammalian or avian species, e.g. primate sp., particularly humans; rodents, including mice, rats and hamsters; rabbits; equines, bovines, canines, felines; etc. Of particular interest are the human HLA proteins, and the murine H-2 proteins. Included in the HLA proteins are the class II subunits HLA-DPα, HLA-DPβ, HLA-DQα, HLA-DQβ, HLA-DRα and HLA-DRβ, and the class I proteins HLA-A, HLA-B, HLA-C, and β 2-microglobulin. Included in the murine H-2 subunits are the class I H-2K, H-2D, H-2L, and the class II I-Aα, I-Aβ, I-Eα and I-Eβ, and β 2-microglobulin. A variety of MHC proteins may be utilized in the subject compositions and methods.

In some embodiments, the MHC protein subunits are a soluble form of the normally membrane-bound protein. The soluble form is derived from the native form by deletion of the transmembrane domain. Conveniently, the protein is truncated, removing both the cytoplasmic and transmembrane domains. The protein may be truncated by proteolytic cleavage, or by expressing a genetically engineered truncated form.

For class I proteins, the soluble form may include the α1, α2 and α3 domains. In some cases, ten or less, such as five or less, or none of the amino acids of the transmembrane domain are included. The deletion may extend as much as about 10 amino acids into the α3 domain. In some instances, none of the amino acids of the α3 domain are deleted. The deletion may be such that it does not interfere with the ability of the α3 domain to fold into a disulfide bonded structure. The class I β-chain, β 2-microglobulin, lacks a transmembrane domain in its native form, and need not be truncated. In some instances, no Class II subunits are used in conjunction with Class I subunits.

Soluble class II subunits may include the α1 and α2 domains for the α-subunit, and the β1 and β2 domains for the β subunit. In some cases, ten or less, such as five or less, or none of the amino acids of the transmembrane domain are included. The deletion may extend as much as about 10 amino acids into the α2 or β2 domain, preferably none of the amino acids of the α2 or β2 domain will be deleted. The deletion will be such that it does not interfere with the ability of the α2 or β2 domain to fold into a disulfide bonded structure.

In some instances, a small number of amino acids are introduced at the polypeptide termini, e.g., not more than 20, more usually not more than 15. The deletion or insertion of amino acids may be as a result of the needs of the construction, providing for convenient restriction sites, addition of processing signals, ease of manipulation, improvement in levels of expression, or the like. In addition, one or more amino acids may be substituted with a different amino acid for similar reasons, e.g., not substituting more than about five amino acids in any one domain.

The alpha and beta subunits may be separately produced and allowed to associate in vitro to form a stable heteroduplex complex (see e.g., Altman et al. (1993, PNAS. 90: 10330-10334) or Garboczi et al. (1992) PNAS. 89:3429-3433) or both of the subunits may be expressed in a single cell. An exchangeable peptide may be originally included on the complex, which is then exchanged with the peptide antigen. An alternative strategy is to engineer a single molecule having both the alpha and beta subunits. A “single-chain heterodimer” is created by fusing together the two subunits using a short peptide linker, e.g. a 15 to 25 amino acid peptide or linker. See Bedzyk et al. (1990) J. Biol. Chem. 265:18615 for similar structures with antibody heterodimers. The soluble heterodimer may also be produced by isolation of a native heterodimer and cleavage with a protease, e.g. papain, to produce a soluble product.

In one embodiment, soluble subunits are independently expressed from a DNA construct encoding a truncated protein. For expression, the DNA sequences are inserted into an appropriate expression vector, where the native transcriptional initiation region may be employed or an exogenous transcriptional initiation region, i.e. a promoter other than the promoter which is associated with the gene in the normally occurring chromosome. The promoter may be introduced by recombinant methods in vitro, or as the result of homologous integration of the sequence into a chromosome. A wide variety of transcriptional initiation regions are known for a wide variety of expression hosts, where the expression hosts may involve prokaryotes or eukaryotes, particularly E. coli, B. subtilis, mammalian cells, such as CHO cells, COS cells, monkey kidney cells, lymphoid cells, particularly human cell lines, and the like. Generally a selectable marker operative in the expression host will be present.

Of particular interest are expression cassettes comprising a transcription initiation region, the gene encoding the subject MHC subunit, and a transcriptional termination region, optionally having a signal for attachment of a poly A sequence. Suitable restriction sites may be engineered into the termini of the MHC subunit, such that different subunits may be put into the cassette for expression. Restriction sites may be engineered by various means, e.g. introduction during polymerase chain reaction, site directed mutagenesis, etc.

The subunits are expressed in a suitable host cell, and, if necessary, solubilized. The two subunits are combined with an antigenic peptide and allowed to fold in vitro to form a stable heterodimer complex with intrachain disulfide bonded domains. The peptide may be included in the initial folding reaction, or may be added to the empty heterodimer in a later step. Usually the MHC binding site will be free of peptides prior to addition of the target antigenic peptide.

The MHC heterodimer may bind the peptide in the groove formed by the two membrane distal domains, either α2 and α1 for class I, or α1 and β1 for class II. The bound peptide may be substantially homogenous, that is, there will be less than about 10% of peptide impurities, such as less than about 5%, or less than about 1%.

Any convenient conditions that permit folding and association of the subunits and peptide may be utilized, see for example Altman et al. (1993) and Garboczi et al. (1992). As one example of permissive conditions, roughly equimolar amounts of solubilized alpha and beta subunits are mixed in a solution of urea. Refolding is initiated by dilution or dialysis into a buffered solution without urea. Peptides are loaded into empty class II heterodimers at about pH 5 to 5.5 for about 1 to 3 days, followed by neutralization, concentration and buffer exchange.

Methods

As summarized above, embodiments of the invention include methods for labeling, detecting or activating an immune cell, e.g. a T cell or B cell, according to specificity of the antigen receptor of the cell, including without limitation a T cell receptor (TCR) or surface antibody (sAb). The subject methods include specifically binding a MHC-peptide spheromer (e.g., as described above) to an antigen receptor. The spheromer may alternatively or in combination comprise a B cell antigen, e.g. a polypeptide, etc.

As such, some aspects of the method include contacting a TCR, generally when present on the surface of a T cell, with a spheromer MHC-peptide complex (e.g., as described above) under conditions by which the spheromer MHC-peptide complex specifically binds the TCR. The TCR-recognition region of the peptide in the complex provides for binding specificity. The spheromer may be free in solution, or may be attached to an insoluble support. Examples of suitable insoluble supports include beads, e.g. magnetic beads, membranes and microtiter plates. In some cases, these may be made of glass, plastic (e.g. polystyrene), polysaccharides, nylon or nitrocellulose. In some cases, the MHC-peptide complex is labeled with a detectable moiety, so as to be directly detectable, or is used in conjunction with secondary labeled reagents which specifically binds the complex.

Any convenient protocol for contacting a cell with the spheromer MHC-peptide complex in a sample may be employed. The particular protocol that is employed may vary, e.g., depending on whether the sample is in vitro or in vivo.

Samples for use in the methods of the invention may be obtained from a variety of sources, particularly blood, although in some instances samples such as bone marrow, lymph, cerebrospinal fluid, synovial fluid, and the like may be used, or cultured T cells. Such samples can be separated by centrifugation, elutriation, density gradient separation, apheresis, affinity selection, panning, FACS, centrifugation with Hypaque, etc. prior to analysis, and often a mononuclear fraction (PBMC) will be used. Once a sample is obtained, it can be used directly, frozen, or maintained in appropriate culture medium for short periods of time.

In some cases, the assay will measure the binding in a patient sample, usually blood derived, generally in the form of plasma or serum and the subject spheromer MHC-peptide complex. The patient sample may be used directly, or diluted as appropriate, usually about 1:10 and usually not more than about 1:10,000. Assays may be performed in any physiological buffer, e.g. PBS, normal saline, HBSS, dPBS, etc.

Various media can be employed to maintain cells. The samples may be obtained by any convenient procedure, such as the drawing of blood, venipuncture, biopsy, or the like. Usually a sample will comprise at least about 10² cells, at least about 10³ cells, at least about 10⁴, 10⁵ or more cells. Often the samples will be from human patients, although animal models may find use, e.g. equine, bovine, porcine, canine, feline, rodent, e.g. mice, rats, hamster, primate, etc. Generally from about 0.001 to 1 ml of sample, diluted or otherwise, is sufficient, usually about 0.01 ml sufficing. The incubation time should be sufficient for the cells to bind the subject complex.

The spheromer is added to a suspension of cells, and incubated for a period of time sufficient to bind the available receptor. The incubation will usually be from about 0.1 to 3 hr, usually 1 hr sufficing. It is desirable to have a sufficient concentration of labeling reagent in the reaction mixture, such that the efficiency of the separation is not limited by lack of reagent. The appropriate concentration can be determined by titration. Various media find use in the labeling. If viable cells are desired, e.g. after a separation procedure, the medium will maintain the viability of the cells, e.g. phosphate buffered saline containing from 0.1 to 0.5% BSA. Various media are commercially available and may be used according to the nature of the cells, including Dulbecco's Modified Eagle Medium (dMEM), Hank's Basic Salt Solution (HBSS), Dulbecco's phosphate buffered saline (dPBS), RPMI, Iscove's medium, PBS with 5 mM EDTA, etc., frequently supplemented with fetal calf serum, BSA, HSA, etc.

Where a second stage labeling reagent is used, the cell suspension may be washed and resuspended in medium as described above prior to incubation with the second stage reagent. Alternatively, the second stage reagent may be added directly into the reaction mix.

The labeled cells can be quantitated as to the expression of a TCR of interest. It is particularly convenient in a clinical setting to perform the immunoassay in a self-contained apparatus. Alternatively various microscopic, flow cytometry, etc. methods find use. Techniques providing accurate enumeration include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g. propidium iodide). Flow cytometry, or FACS, can also be used to separate cell populations based on the intensity of fluorescence, as well as other parameters.

Flow cytometry may also be used for the separation of a labeled subset of T cells from a complex mixture of cells. The cells may be collected in any appropriate medium which maintains the viability of the cells, usually having a cushion of serum at the bottom of the collection tube. Various media are commercially available as described above. The cells may then be used as appropriate.

Alternative means of separation utilize the subject complex bound directly or indirectly to an insoluble support, e.g. column, microtiter plate, magnetic beads, etc. The cell sample is added to the binding complex. The complex may be bound to the support by any convenient means. After incubation, the insoluble support is washed to remove non-bound components. From one to six washes may be employed, with sufficient volume to thoroughly wash non-specifically bound cells present in the sample. In particular the use of magnetic particles to separate cell subsets from complex mixtures is described in Miltenyi et al. (1990) Cytometry 11:231-238.

The insoluble supports may be any compositions to which the multimeric binding complex can be bound, which is readily separated from soluble material, and which is otherwise compatible with the overall method of measuring T cells.

Generally the number of bound T cells detected will be compared to control samples from samples having a different MHC context or antigen specificity, e.g. T cells from an animal that does not express the MHC molecule used to make the binding complex.

T Cells. The subject spheromer MHC-peptide complexes (labeling reagent) and methods are used to activate, label, detect and/or separate antigen specific T cells. The T cells may be from any source, usually having the same species of origin as the MHC molecules. The T cells may be from an in vitro culture, or a physiologic sample. In many cases, the physiologic samples employed will be blood or lymph, but samples may also involve other sources of T cells, particularly where T cells may be invasive. Thus, other sites of interest are tissues, or associated fluids, as in the brain, lymph node, neoplasms, spleen, liver, kidney, pancreas, tonsil, thymus, joints, synovia, and the like. The sample may be used as obtained or may be subject to modification, as in the case of dilution, concentration, or the like. Prior treatments may involve removal of cells by various techniques, including centrifugation, using Ficoll-Hypaque, panning, affinity separation, using antibodies specific for one or more markers present as surface membrane proteins on the surface of cells, or any other technique that provides enrichment of the set or subset of cells of interest.

TCR specificity determines what antigens will activate that particular T cell. In general terms, T helper cells express CD4 on their surface, and are activated by binding to a complex of antigenic peptide and Class II MHC molecule. Cytolytic T cells may express CD8 on their surface, and are activated by binding to a complex of antigenic peptide and Class I MHC molecule. The specificity of the T cell antigen receptor is the combination of peptide and MHC molecule that binds to that particular TCR with sufficient affinity to activate the T cell. A variety of MHC-peptide complexes may be utilized in the binding complex. Complexes of class I MHC molecules may be used to detect CD8+ T cells, and class II complexes may be used to detect CD4+ T cells. Quantitation of T cells may be performed to monitor the progression of a number of conditions associated with T cell activation, including autoimmune diseases, graft rejection, vital infection, bacterial and protozoan infection. T cells having a particular antigenic specificity may be separated from complex mixtures, particularly biological samples, utilizing the subject methods. In this way selective depletion or enrichment of particular T cells can be made.

Utility. The spheromer MHC-peptide complexes, peptides and methods of the invention, e.g., as described above, find use in a variety of applications. Applications of interest include, but are not limited to: research applications, diagnostic applications and therapeutic applications. Methods of the invention find use in a variety of different applications including any convenient application related to the detection and/or purification of T cells. In such cases, the subject MHC-peptide complexes and methods may be used to activate, label, detect and/or separate a T-cell of interest in a sample.

The subject spheromer MHC-peptide complexes and methods find use in a variety of diagnostic applications, including but not limited to, the diagnosis of a disease condition associated with the T-cell, e.g., in vitro diagnostics or in vivo diagnostics. Such applications are useful in diagnosing or confirming diagnosis of a disease condition, or susceptibility thereto, determining the proper course of treatment for a patient suffering from a disease condition. The methods are also useful for monitoring disease progression and/or response to treatment in patients who have been previously diagnosed with the disease. Diagnostic applications of interest include diagnosis of disease conditions, including but not limited to: tumors, infectious diseases and autoimmune diseases.

Detection of T cells is of interest in connection with a variety of conditions associated with T cell activation. Such conditions include autoimmune diseases, e.g. multiple sclerosis, myasthenia gravis, rheumatoid arthritis, type 1 diabetes, graft vs. host disease, Grave's disease, etc.; various forms of cancer, e.g. carcinomas, melanomas, sarcomas, lymphomas and leukemias. Various infectious diseases such as those caused by viruses, e.g. HIV-1, hepatitis, herpesviruses, enteric viruses, respiratory viruses, rhabdovirus, rubeola, poxvirus, paramyxovirus, morbillivirus, etc. are of interest. Infectious agents of interest also include bacteria, such as Pneumococcus, Staphylococcus, Bacillus. Streptococcus, Meningococcus, Gonococcus, Eschericia, Klebsiella, Proteus, Pseudomonas, Salmonella, Shigella, Hemophilus, Yersinia, Listeria, Corynebacterium, Vibrio, Clostridia, Chlamydia, Mycobacterium, Helicobacter and Treponema; protozoan pathogens, viral infections including, for example, corovavirus infections, and the like. T cell associated allergic responses may also be monitored, e.g. delayed type hypersensitivity or contact hypersensitivity involving T cells.

Also of interest are conditions having an association with a specific peptide or MHC haplotype, where the subject complexes may be used to track the T cell response with respect to the haplotype and antigen. A large number of associations have been made in disease states that suggest that specific MHC haplotypes, or specific protein antigens are responsible for disease states. In such cases, direct detection of reactive T cells in patient samples is of interest. Detection and quantitation with the subject complexes allows such direct detection. As examples, the activity of cytolytic T cells against HIV infected CD4+ T cells may be determined using the subject methods. The association of diabetes with the DQB1*0302 (DQ3.2) allele may be investigated by the detection and quantitation of T cells that recognize this MHC protein in combination with various peptides of interest. The presence of T cells specific for peptides of myelin basic protein in conjunction with MHC proteins of multiple sclerosis patients may be determined. The antigenic specificity may be determined for the large number of activated T cells that are found in the synovial fluid of rheumatoid arthritis patients. It will be appreciated that the subject methods are applicable to a number of diseases and immune-associated conditions.

Exemplified herein are SARS-CoV-2 peptides associated with MHC HLA-A*02:01. These peptides can be used to map specific virus epitopes that generate a T cell response of interest.

The isolation of antigen specific T cells finds a wide variety of applications, including therapeutic applications. The isolated T cells may find use in the treatment of cancer as in the case of tumor-infiltrating lymphocytes. Specific T cells may be isolated from a patient, expanded in culture by cytokines, antigen stimulation, etc., and replaced in the autologous host, so as to provide increased immunity against the target antigen. A patient sample may be depleted of T cells reactive with a specific antigen, to lessen an autoimmune response.

The DNA sequence of single T cell receptors having a given antigen specificity is determined by isolating single cells by the subject separation method. Conveniently, flow cytometry may be used to isolate single T cell, in conjunction with single cell PCR amplification. In order to amplify unknown TCR sequences, various amplification protocols may be used.

In some embodiments kits are provided, where the kits include one or more components employed in methods of the invention, e.g., modified ferritin proteins, peptides, MHC molecules, MHC-peptide complexes, T cells, and the like. In some embodiments, the subject kit includes a spheromer MHC-peptide particle (e.g., as described herein), or precursor components thereof, and one or more components selected a T cell, a buffer, instructions, and the like. The subject kits may further comprise additional reagents which are required for or convenient and/or desirable to include in the reaction mixture prepared during the subject methods, where such reagents include buffers for specifically binding complexes to T cells; reagents, and the like.

Kits may also include tubes, buffers, etc., and instructions for use. The various reagent components of the kits may be present in separate containers, or some or all of them may be pre-combined into a reagent mixture in a single container, as desired.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another form of these instructions is a computer readable medium, e.g., diskette, compact disk (CD), etc., on which the information has been recorded. Yet another form of these instructions that may be present is a website address which may be used via the internet to access the information at a removed site.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Improved Staining Reagent Shows Conserved SARS-CoV-2 Peptide-Specific T Cells Correlate with Milder Disease

A central feature of the SARS-CoV-2 pandemic is that some individuals become severely ill or die, whereas others have only a mild disease course or are asymptomatic. Here we have developed a superior αβ T cell staining reagent, with each maxi-ferritin “spheromer” displaying 12 peptide-MHC complexes. This stains specific T cells more efficiently and captures a broader portion of the sequence repertoire for a given peptide-MHC. Analyzing T cells from unexposed individuals, we find that peptides conserved amongst coronaviruses are more abundant and tend to have a “memory” phenotype, compared to those unique to SARS-CoV-2. Significantly, CD8+ T cells with these conserved specificities are much more abundant in COVID-19 patients with mild disease versus those with a more severe illness, suggesting a protective role.

Severe acute respiratory syndrome coronavirus 2 (SARS-CoV-2), the virus causing COVID-19 has infected ˜50 million individuals worldwide, displaying a spectrum of disease severities that ranges from asymptomatic to life-threatening pneumonia and multi-organ failure. Addressing this global pandemic, many pharmaceutical companies and research laboratories have raced to develop effective coronavirus vaccines, of which over a hundred are in development. The primary endpoint in most studies is the generation of neutralizing antibodies targeting the SARS-CoV-2 spike (S) protein. However, the variable magnitude and durability of these antibody responses in COVID-19 patients highlights the importance of studying T cell mediated immunity to better understand disease pathogenesis and to develop benchmarks for an effective T cell response. Many studies have shown that T cells are involved in a SARS-CoV-2 infection, but what types of responses are efficacious and which are not is unclear.

In order to improve upon the limitations of pMHC staining, we engineered a biotinylation site on maxi-ferritin to create a 24-subunit self-assembling protein scaffold for the multivalent display of pMHC. This “spheromer” platform offers several advantages: ease of production, defined site-specific conjugation of pMHC molecules that significantly reduces inter-batch variation, and compatibility with currently available pMHC molecules and streptavidin reagents that allows for facile translation. We show that the spheromer binds both MHC-I and MHC-II restricted T cells with excellent specificity for pMHC, and at a significantly higher avidity than the tetramer. Furthermore, this reagent provides a better signal-to-noise ratio and detects a much more diverse antigen-specific TCR repertoire in comparison to equivalent tetramers or dextramers. Finally, using the spheromer for direct ex vivo study of SARS-CoV-2 specific CD8⁺ T cells, we show that T cells predicted to cross-react with seasonal human coronaviruses are significantly enriched in COVID-19 patients with mild symptoms in comparison to individuals with severe disease. Since there is evidence that antibodies to SARS-CoV-2 wane not long after infection, these robust T cells to conserved epitopes detected in SARS-CoV-2 unexposed individuals and in those with mild disease could be the key determinant in a successful adaptive immune response and could help to explain the disparity COVID-19 outcomes. Furthermore, following these T cells using spheromer technology could help in tracking SARS-CoV-2 vaccine immunity in vaccinated individuals.

Results

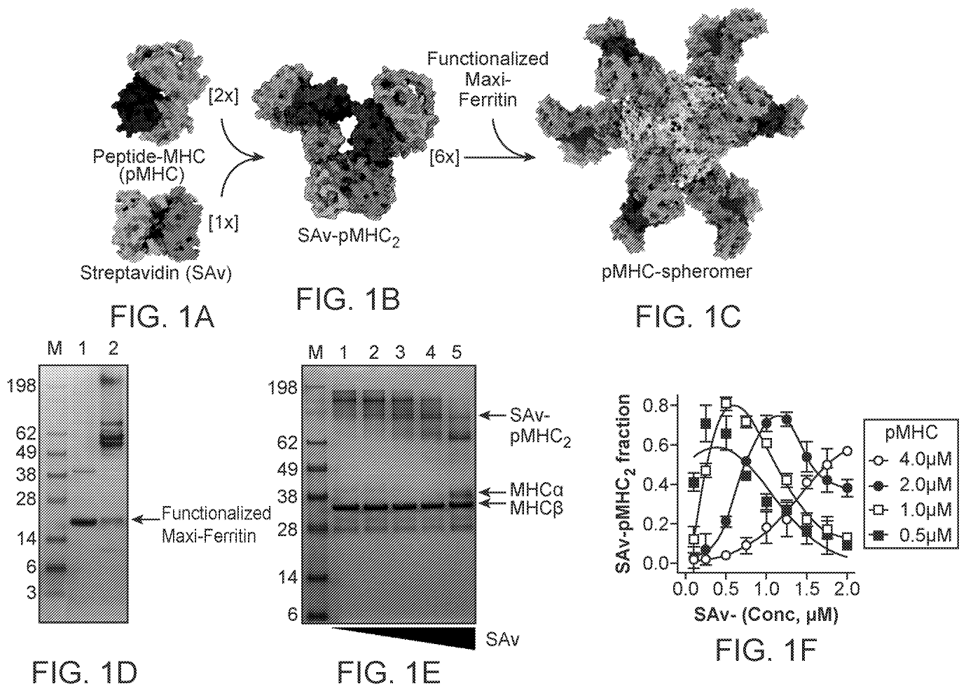

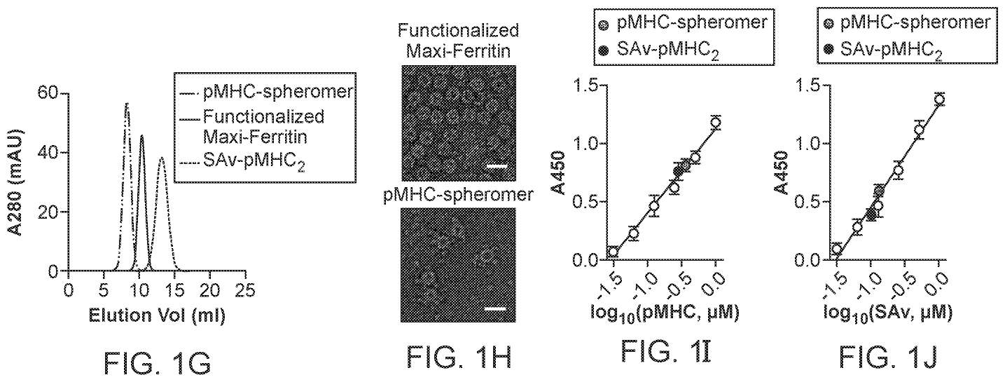

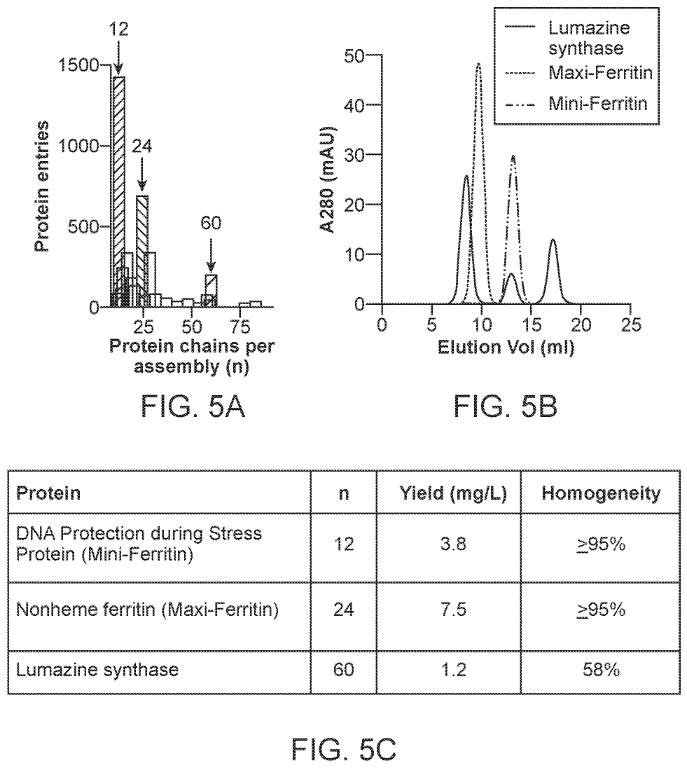

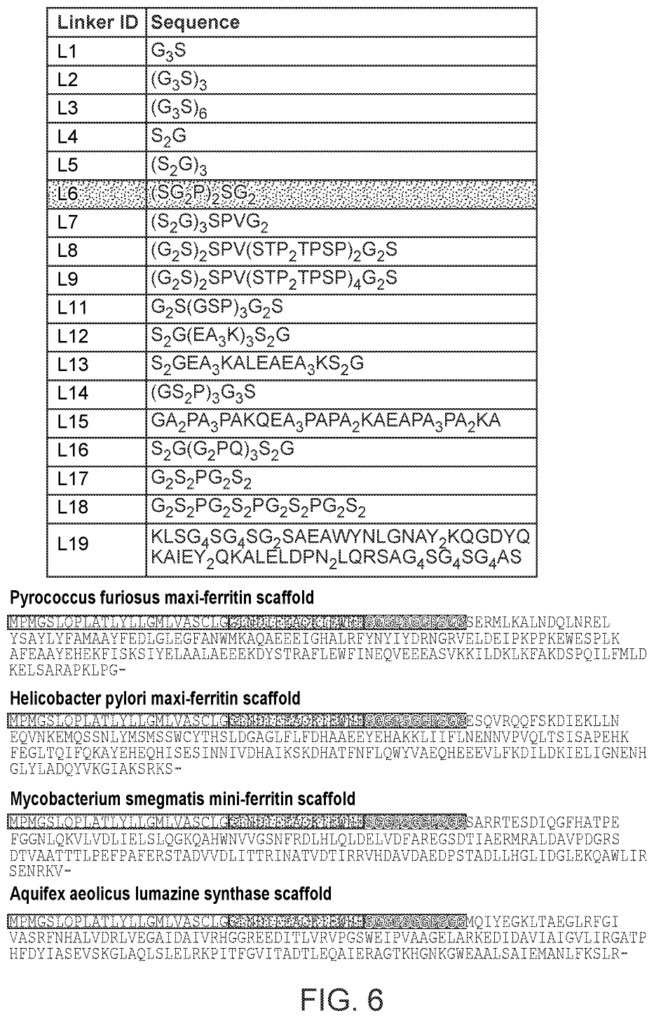

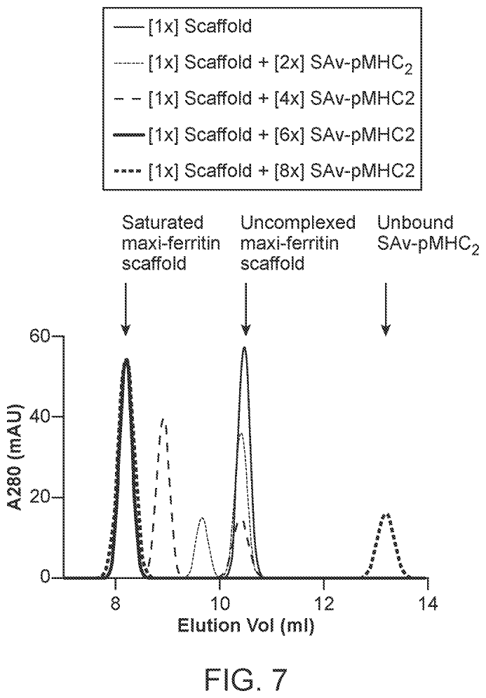

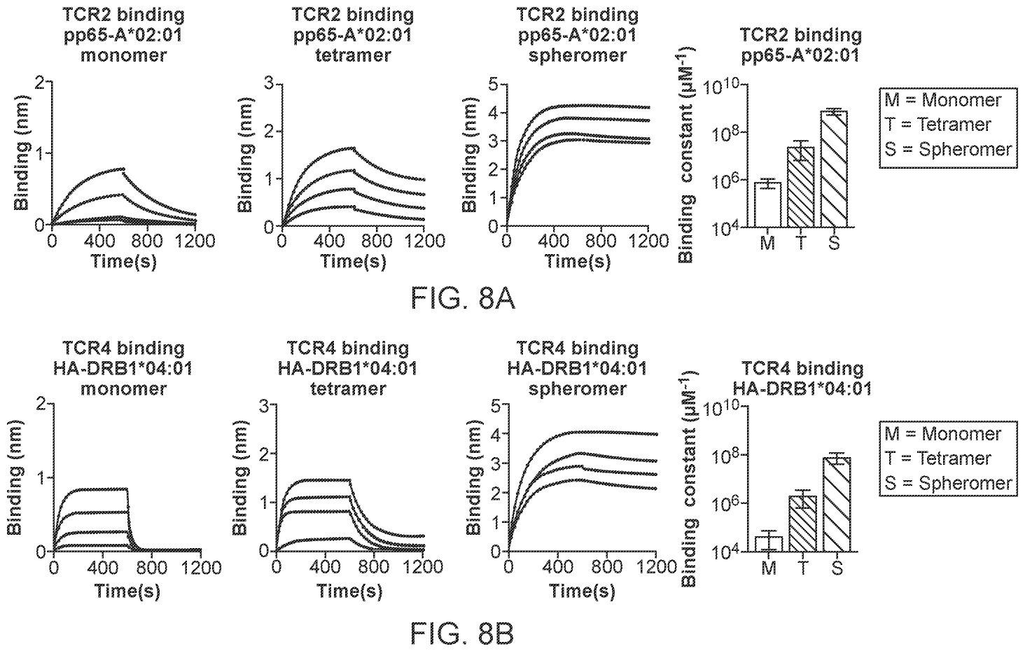

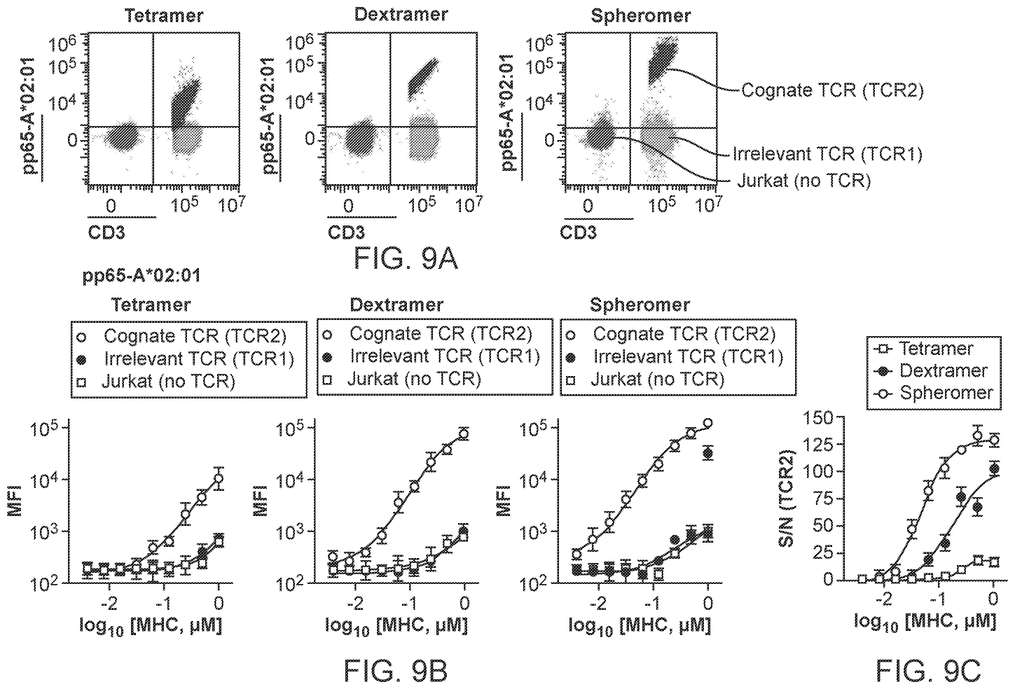

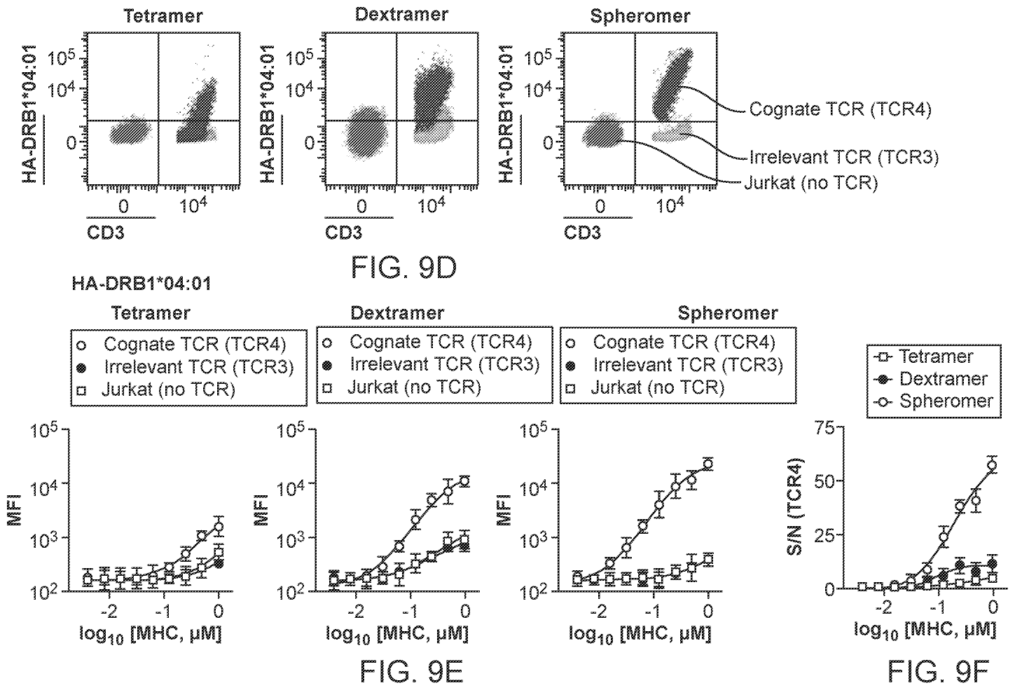

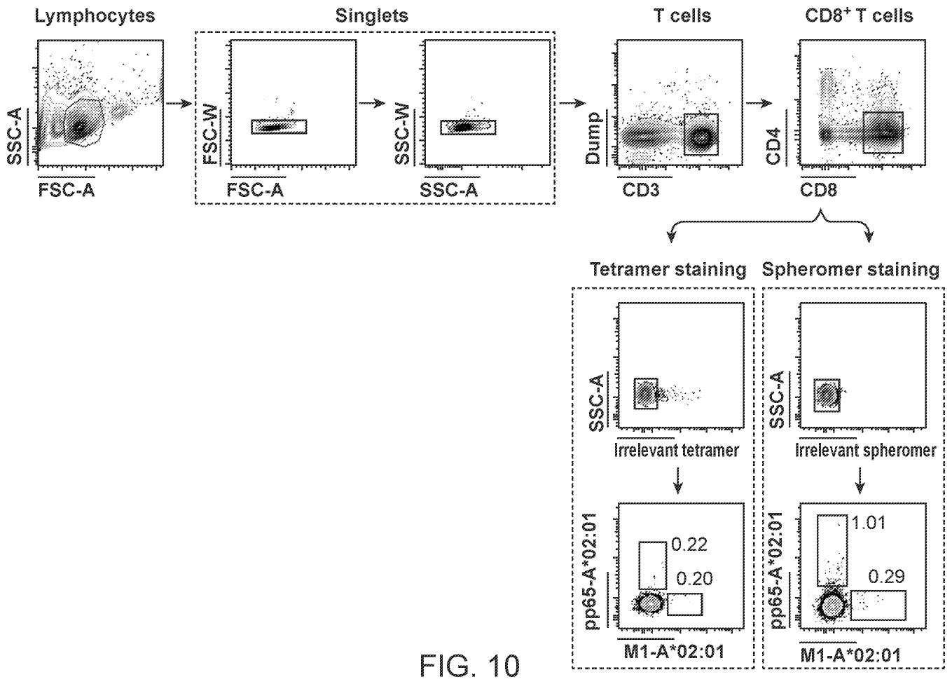

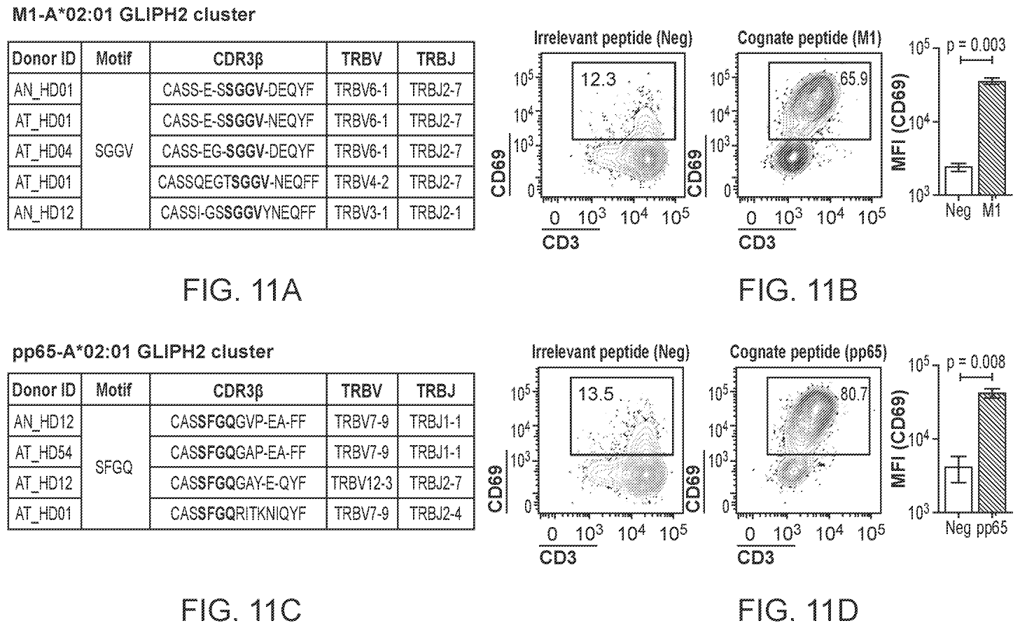

In the search for a protein scaffold that could increase the valency of displayed pMHC and that would capture more αβ T cells of a given specificity, we focused on self-assembling homo-oligomers (FIG. 5A). Based on the yield and homogeneity of the recombinantly expressed proteins (FIG. 5B-C), we chose maxi-ferritin for further optimization. Ferritins are naturally occurring cage proteins that participate in biomineral synthesis and are found across almost all living organisms. Studies have shown that thermophilic proteins denature at a much higher temperature than their mesophilic homologs. Therefore, we used ferritin derived from the hyperthermophilic archaeal anaerobe Pyrococcus furiosus to develop a stable scaffold. Maxi-ferritin forms a 24-subunit nanoparticle with an external diameter of ˜120 Å. In order to develop a platform that is widely accessible, we functionalized the maxi-ferritin scaffold to be compatible with components of the existing tetramer technology that uses biotinylated pMHC monomers and SAv conjugates. We inserted a biotinylation signal sequence at the N-terminus of each maxi-ferritin subunit (˜23 kDa monomer) and utilized SAv as a ‘molecular glue’ to bring together pMHC monomers and the scaffold (FIG. 1A-C). We optimized the tethers for SAv on the maxi-ferritin scaffold by testing a set of linkers that spanned a diverse range of lengths and molecular rigidities (FIG. 6A-B)(25). As shown, the optimized scaffold with radially projecting tethers could be purified easily and functionalized with biotin (FIG. 1D). We then bound the biotinylated scaffold to SAv conjugated to two peptide-MHC molecules (SAv-pMHC₂: semi-saturated SAv) (FIGS. 1E-F). The semi-saturated SAv has two biotin binding sites available to bind the scaffold. Upon saturation, we observed the display of 12 pMHC molecules as determined by size-exclusion chromatography (SEC) (FIG. 7). The current prototype does not allow the conjugation of more pMHC molecules, presumably because of steric hindrance. We further purified the homogeneous spheromer by size-exclusion chromatography to exclude the contribution from any unreacted SAv-pMHC₂ (FIG. 1G). We also validated the conjugation of SAv-pMHC₂ onto the functionalized maxi-ferritin scaffold using negative-stain EM (FIG. 1H) and ELISA (FIG. 1I-J).

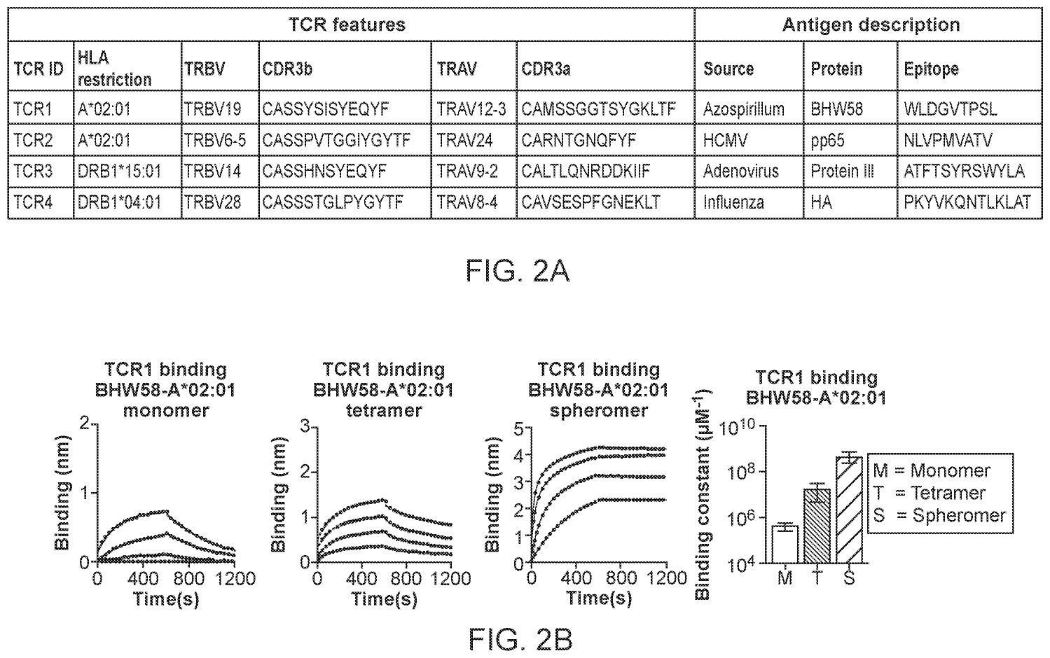

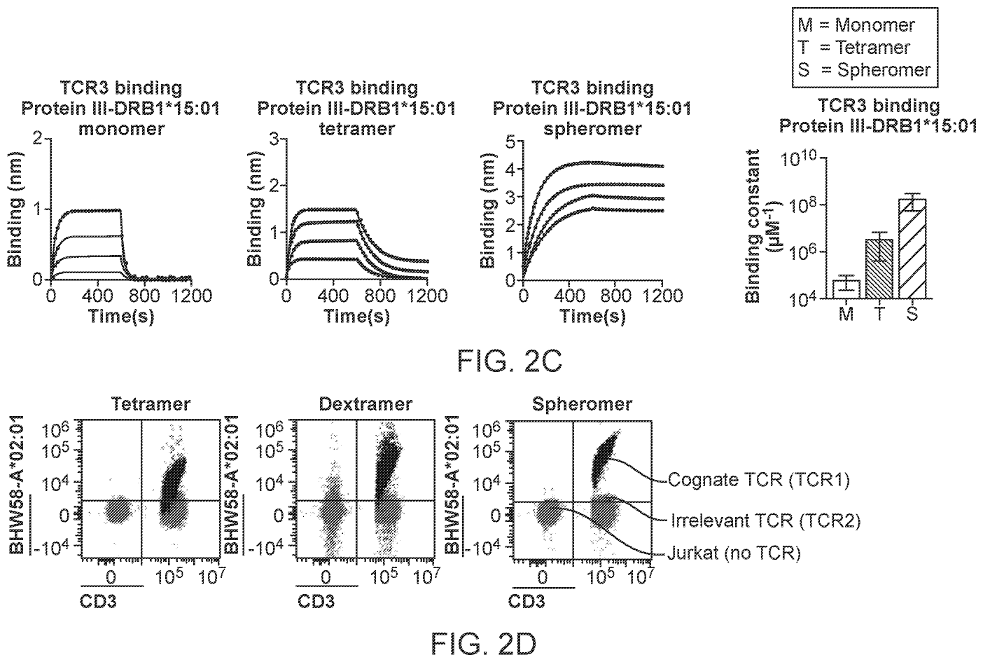

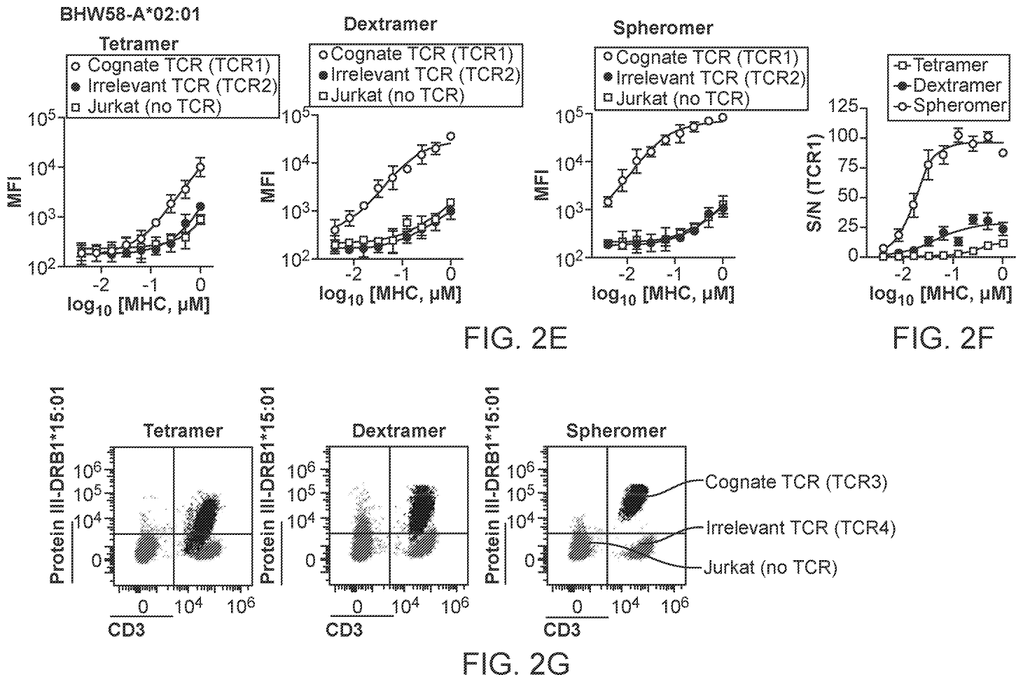

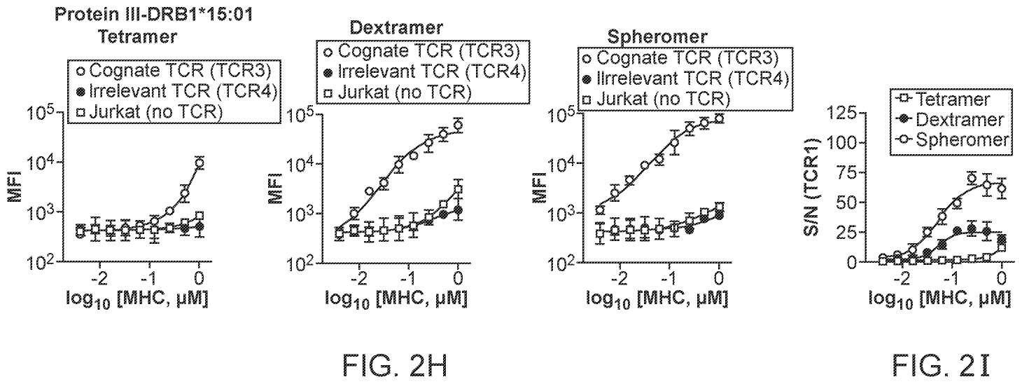

We characterized the general applicability of the spheromer using a set of TCR-pMHC pairs with distinct TRBV usage, antigen sources, and examples representing both MHC-I and MHC-II molecules (FIG. 2A). The binding of TCR with different formulations of their cognate pMHC (monomer, tetramer and spheromer) was determined using biolayer interferometry (FIGS. 2B-C and 8A-B). Encouragingly, the spheromer bound all the evaluated TCRs significantly better than the other formulations (monomer and tetramer). On average, for MHC-I restriction, the spheromer bound TCRs with >250 (monomer) and >50-fold (tetramer) greater net-affinity. While, for MHC-II restricted TCRs, the spheromer bound with >200 (monomer) and >20-fold (tetramer) greater net-affinity across the tested pairs. We also generated stable T cell lines to compare the binding of different pMHC formulations (tetramer, dextramer and spheromer) using flow cytometry (FIGS. 2D-I and 9A-F). As shown, for all the evaluated pMHC-TCR pairs, consistent with the increased avidity, the signal from spheromer staining was significantly better (˜10-fold) than the tetramer. We included negative controls (TCR⁻ Jurkat cells and a cell line expressing irrelevant TCR) to determine background staining since higher-valency can result in noise amplification due to non-specific interactions. We observed that while there was an increase in staining intensity with dextramer staining (˜6-fold) in comparison to the tetramer, the background staining was also higher. In contrast, the background staining with the spheromer did not increase substantially, resulting in a better signal-to-noise ratio compared to other pMHC-formulations (FIGS. 2F, I and 9C, F). This difference is likely because the spheromer is a discrete, homogenous structure versus a mix of dextran polymers in the dextramer reagents.

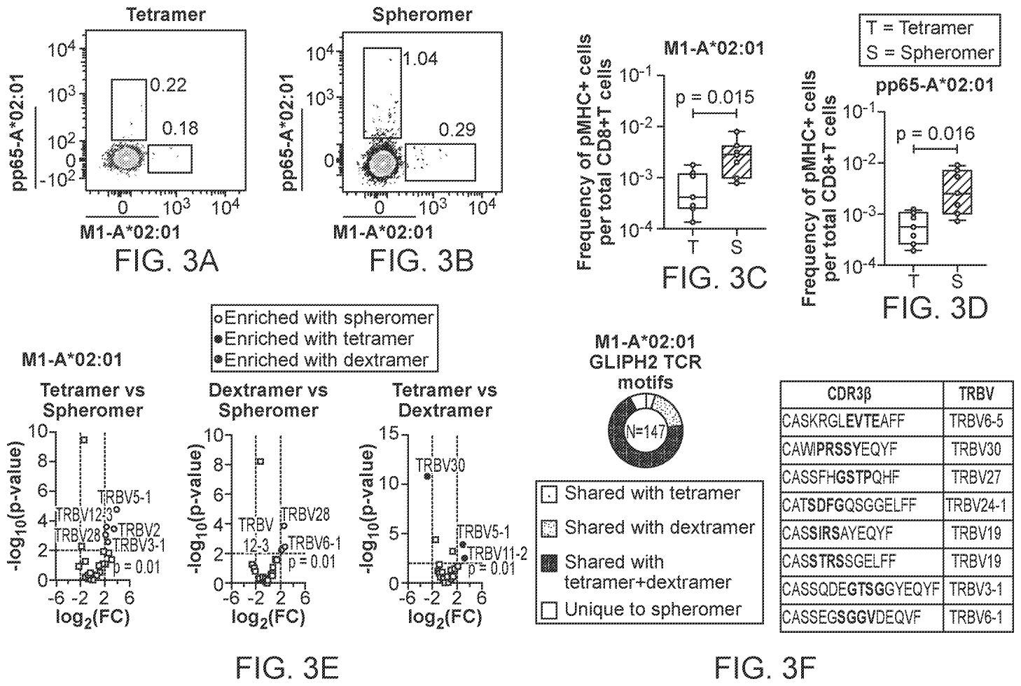

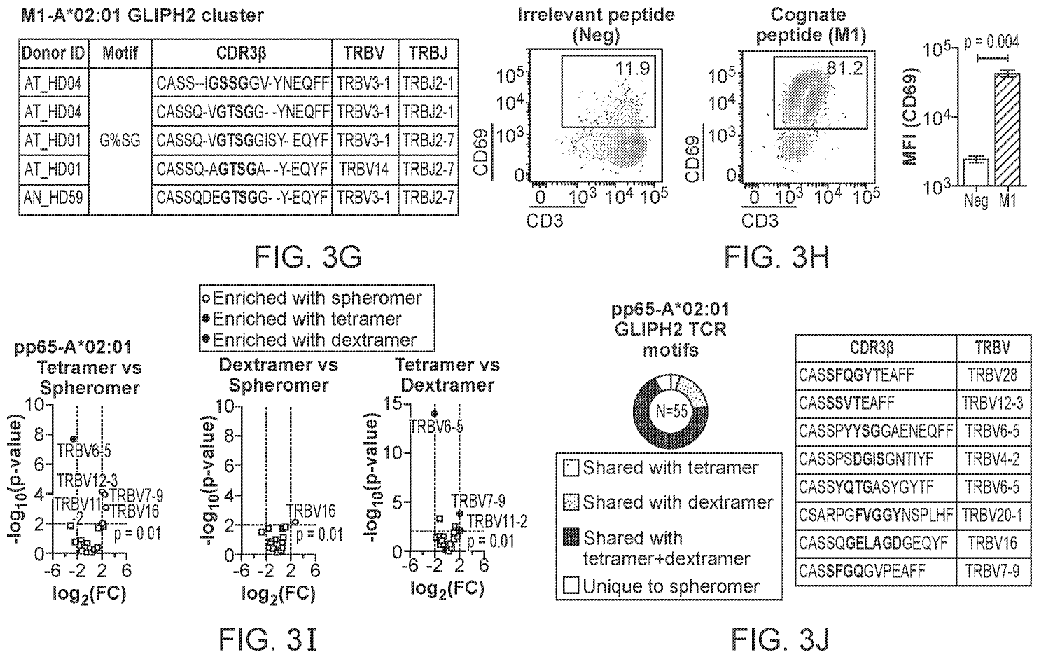

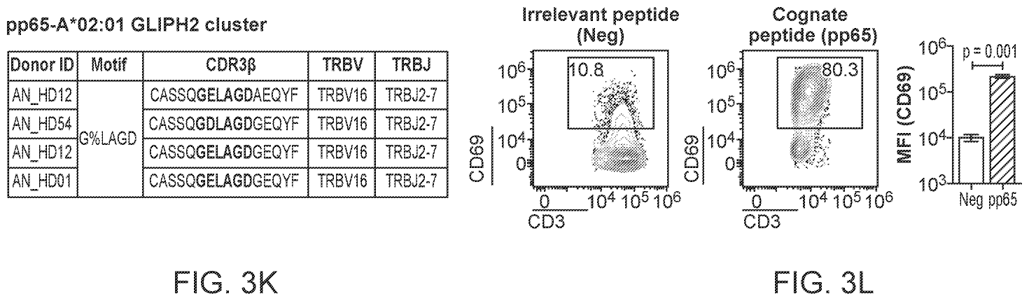

Next, we evaluated viral-specific CD8⁺ T cells in healthy individuals to address the following questions: i) Does the spheromer detect a higher frequency of antigen-specific T cells than tetramer ex vivo? ii) How do the TCR repertoires detected by the spheromer and tetramer compare? We used immunodominant HLA-A*02:01 restricted epitopes (influenza-M1 and HCMV-pp65) for characterizing the spheromer since there is considerable data available for benchmarking. CD8⁺ T cells isolated from each donor (n=7) were divided evenly for tetramer or spheromer staining (FIGS. 3A-B, 10). As shown, a significantly higher frequency of antigen-specific CD8⁺ T cells could be detected for both M1 (p=0.015) and pp65 (p=0.016) viral specificities (FIG. 3C-D). We single-cell sorted spheromer⁺ CD8⁺ T cells and performed paired αβ-TCR sequencing to study the repertoire. The spheromer derived TCR sequences were analyzed against TCR entries in VDJdb, a curated database of TCRs with known antigen specificities. We compared the TRBV usage of TCR sequences obtained using distinct pMHC formulations (FIG. 3E, I). Overall, we observed that the spheromer detected a much more diverse repertoire in comparison to either the tetramer or dextramer. As shown, the M1-specific TCR sequences detected with the spheromer had a significantly (p-value<0.01, fisher's test) higher usage of 5 and 3 TRBV genes in comparison to the tetramer and dextramer derived sequences, respectively, with 2 overlapping genes (TRBV12-3 and TRBV28) across them (FIG. 3E). Similarly, spheromer⁺ pp-65 TCR sequences showed an enrichment of 4 TRBV genes in comparison to the tetramer and 1 TRBV gene with the dextramer (FIG. 3I). Intriguingly, TRBV6-5 is significantly enriched in tetramer⁺ pp-65⁺ TCR sequences when compared to both the dextramer and spheromer derived sequences. We further analyzed the specificity of spheromer derived TCR sequences using GLIPH2 (grouping of lymphocyte interaction by paratope hotspots), an algorithm that clusters TCR based on shared antigen specificity (FIG. 3F, J). Globally, we observed a significant overlap (˜91%) between the TCR ‘motifs’ identified using spheromer and antigen-specific TCR entries in VDJdb. The recovery of previously characterized antigen-specific TCR motifs using the spheromer provides further confirmation that our designed platform is indeed detecting relevant T cells. The spheromer could detect previously described public TCRs for both M1 (CDR3β: CASSIRSSYEQYF, CASSIRSAYEQYF) and pp65 (CDR3β: CASSYQTGASYGYTF) viral specificities shown to have a significant association with HLA-A*02:01. Interestingly, the spheromer identified novel TCR motifs (M1-8% and pp65-9%) that did not cluster with sequences previously reported in VDJdb. In order to test whether these TCRs could confer reactivity to the pMHCs they were selected with, we generated T cell lines with TCRs from these novel GLIPH2 clusters (FIGS. 3G, K and 11A, C). As shown using CD69 expression, these T cell lines could be activated specifically using the cognate peptide (FIGS. 3H, L and 11B, D). These results demonstrate that spheromer reagents are not just more efficient at staining the relevant T cells, but also can identify antigen-specific T cells that may be lost with other multimer reagents.

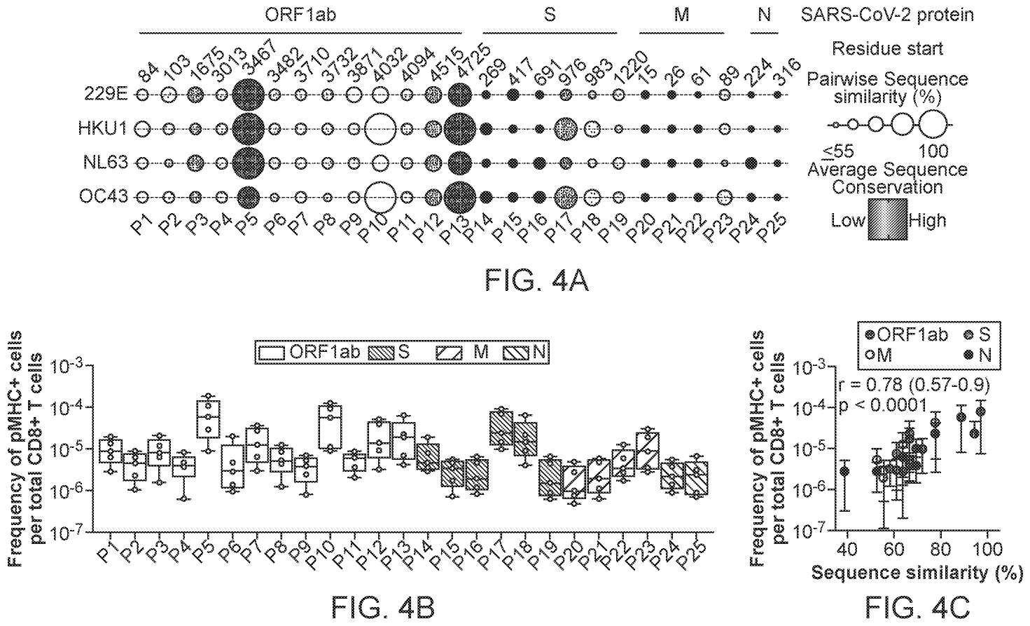

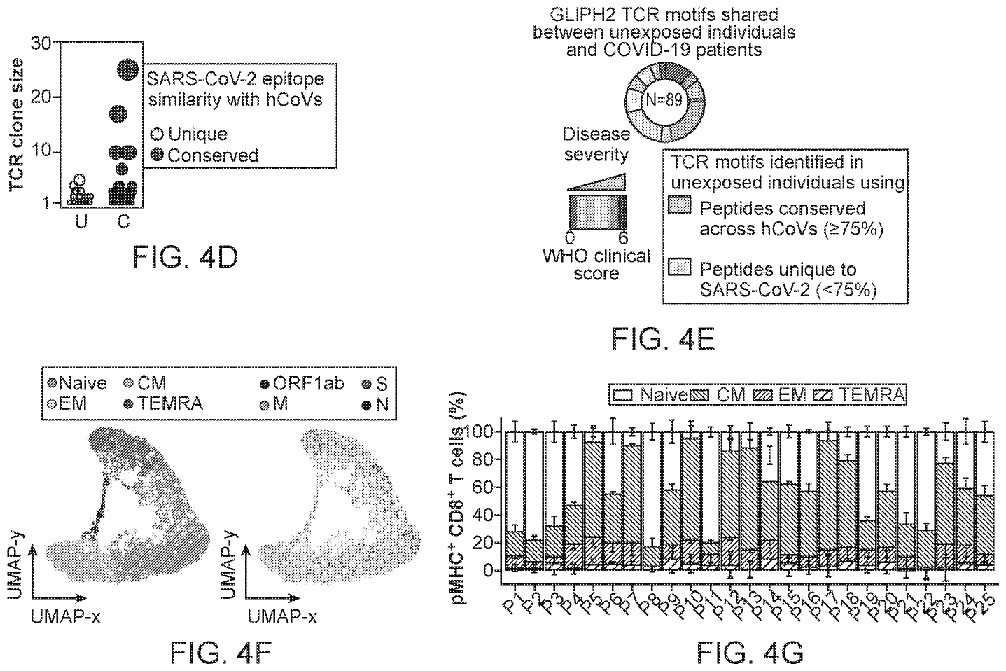

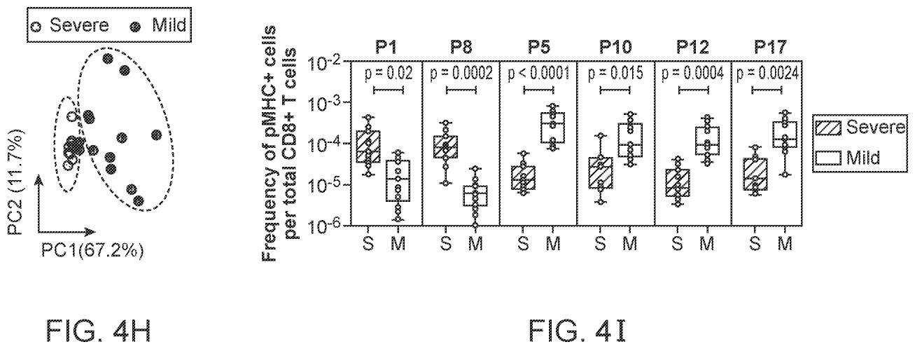

To address the immune response to SARS-CoV-2, we made spheromer reagents to evaluate CD8⁺ T cell responses in unexposed individuals and COVID-19 patients. We have previously shown that T cells to viral epitopes can be detected in the peripheral blood of naïve individuals. Significantly, a large fraction (˜50%) of these T cells in adults (28-80 y) exhibited a memory phenotype, possibly due to higher TCR cross-reactivity or environmental exposures. The rapid recruitment of these T cells in an immune response could offer a survival advantage, since clonal expansion and the induction of memory lymphocytes is a key goal of vaccination efforts and strongly correlates with protection against particular infectious diseases. Previous studies have also shown that T cell precursor frequencies correlate with the magnitude of anti-viral responses. Therefore, we determined the frequency of CD8⁺ T cells against a panel of SARS-CoV-2 epitopes in naïve, unexposed individuals using the spheromer (FIG. 4A).

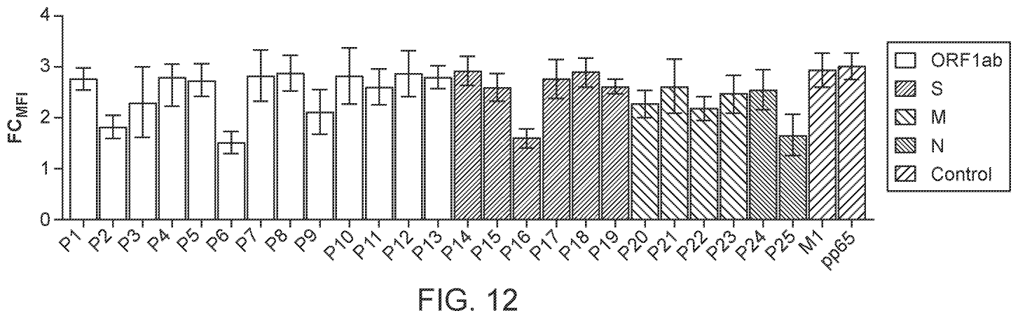

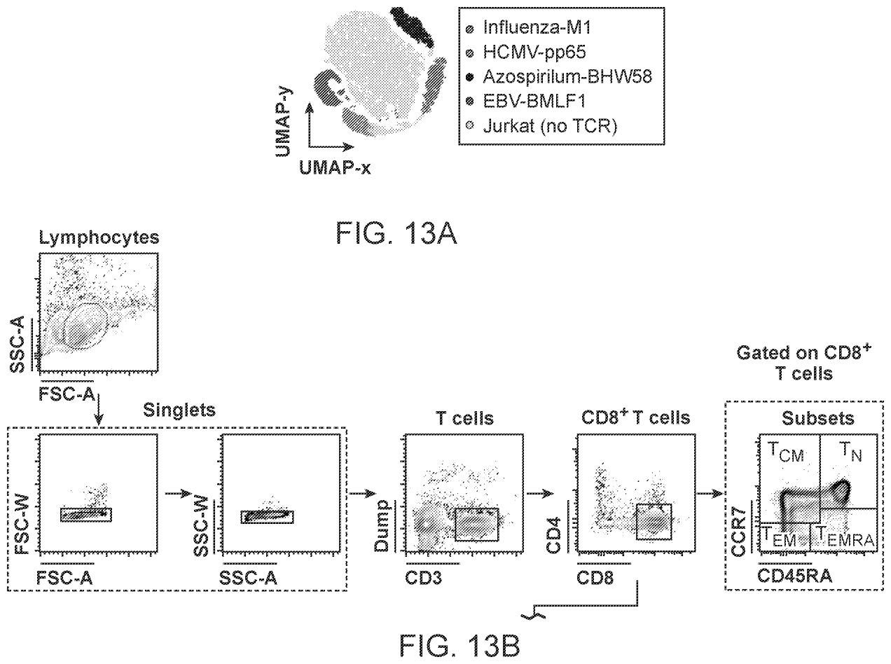

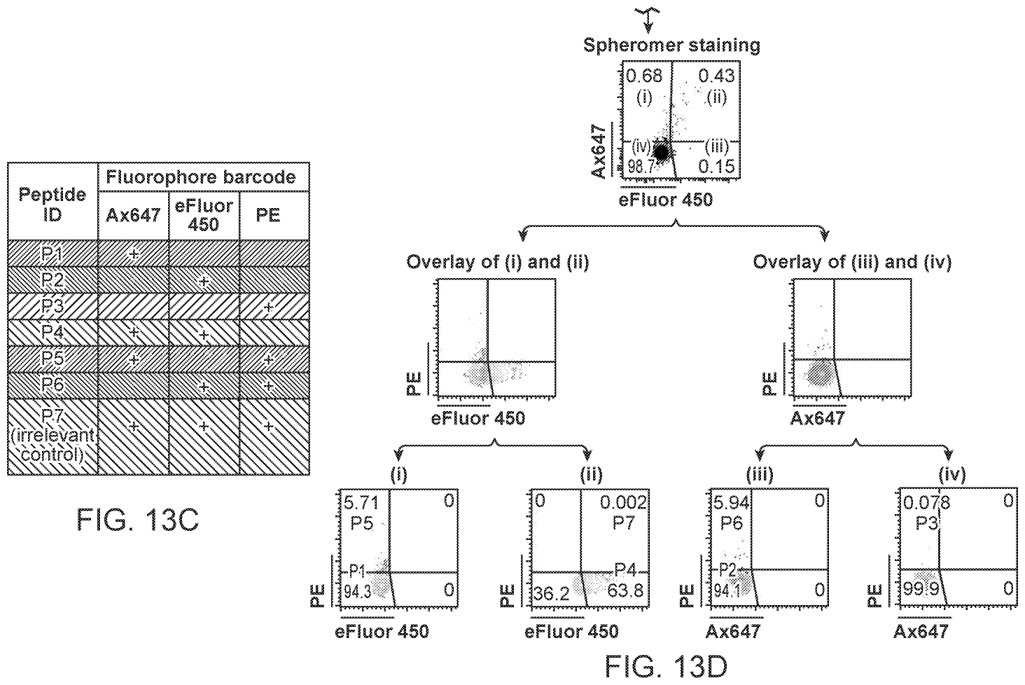

The peptides were selected across different SARS-CoV-2 open reading frames (ORFs), taking into consideration their biochemical properties and their predicted binding affinity to HLA-A*02:01 (Table S1). We used an MHC stabilization assay to further validate the binding of peptides to A*02:01 MHC-I molecules expressed on a T2 cell line (FIG. 12). Also, we designed our peptide panel to represent a diverse range of sequence similarities with peptides from common cold-causing human coronaviruses (hCoV-OC43, HKU1, 229E, NL63) to evaluate cross-reactive responses. We used a combinatorial staining approach as described previously to simultaneously probe for multiple specificities in a single sample (FIG. 13).

In unexposed individuals, we observed that a few SARS-CoV-2 epitopes (P5, P10, P12, P13, P17 and P18) had an elevated CD8⁺ T cell frequency (2.07×10⁻⁴±1.16×10⁻⁴) when compared to other peptides (2.96×10⁻⁵±2.01×10⁻⁵) in the panel (FIG. 4B). Generally, epitopes to which we observed elevated T cell frequencies were characterized by high sequence similarity with hCoVs (FIG. 4C). TCR sequencing of CD8⁺ T cells from unexposed individuals using spheromer presenting SARS-CoV-2 epitopes showed that T cells identified using peptides conserved across coronaviruses are relatively expanded in comparison to T cells against peptides unique to SARS-CoV-2 (FIG. 4D). Furthermore, using GLIPH2 we could identify TCR motifs shared between unexposed individuals and COVID-19 patients (FIG. 4E). This suggests that T cells found in unexposed individuals that bind SARS-CoV-2 epitopes could be actively recruited during infection. Also, TCR motifs against conserved epitopes are enriched in COVID-19 patients with mild symptoms.

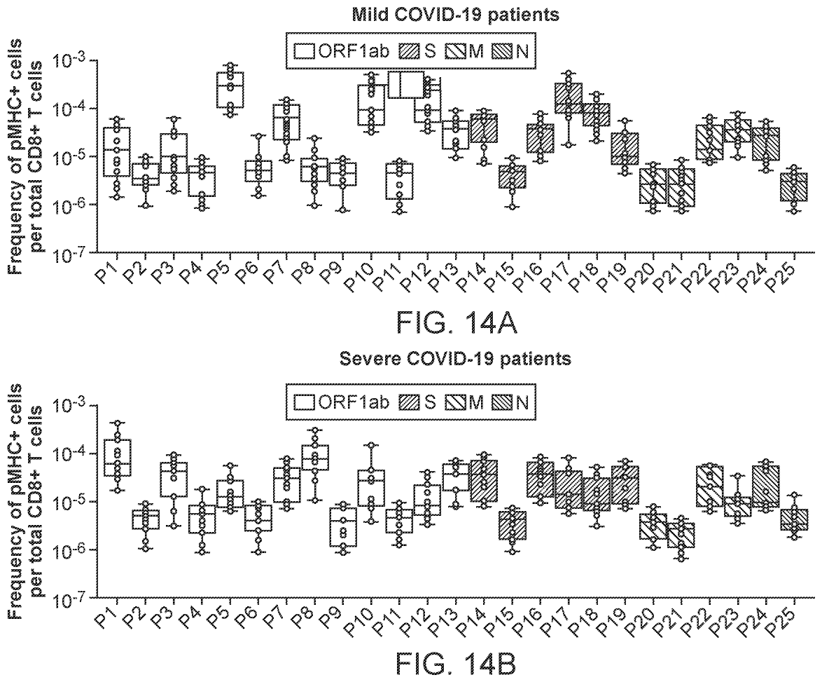

In contrast, TCR motifs characterizing severe COVID-19 patients were detected using peptides that were primarily unique to SARS-CoV-2. Phenotypic characterization of these antigen-specific T cells using CCR7 and CD45RA markers showed a distinct distribution between the naïve/memory compartments for the tested peptides (FIG. 4F-G). T cells detected with peptides having low hCoV sequence similarity demonstrated a predominantly naïve phenotype. In contrast, peptides against which relatively elevated T cell frequencies were observed in unexposed individuals showed a memory phenotype (˜80%) and correlated with high hCoV sequence similarity. This suggests that exposure to seasonal hCoVs among other cross-reactive environmental exposures could contribute to the observed expansion of these T cells. Next, we determined the CD8⁺ T cell frequencies for SARS-CoV-2 epitopes in COVID-19 patients presenting mild or severe symptoms (FIG. 14). The frequency of CD8⁺ T cells demonstrated a distinct group-specific pattern as shown in the principal component analysis (PCA) (FIG. 4H). Intriguingly, the peptides (P1 and P8) which showed a higher response in severe patients, have a low similarity to other hCoVs. In contrast, patients exhibiting mild symptoms showed an elevated response to peptides (P5, P10, P12 and P17) which have a high sequence similarity to other hCoVs (FIG. 4I). Overall, our data show a preferential recruitment of memory CD8⁺ T cells specific for conserved epitopes, that are likely the result of previous hCoV exposures in COVID-19 patients developing mild symptoms.

Antigen-specific T cell responses are known to be essential for an effective immune response against many infectious diseases but defining specific benchmarks for what is protective versus what is not has been challenging, especially in human studies. This is due to many factors, including the low frequency of disease-relevant T cells, particularly when clinical samples are limiting, as they typically are. Consequently, some methods used to investigate T cells necessitate expansion of cells in culture which may alter the relative abundance and phenotype of some T cell clonotypes. Also, the TCR repertoire cannot be studied with some of these methods due to their incompatibility with sequencing techniques. The development of tetramer technology partially addressed this limitation and enabled the direct measurement and characterization of T cells ex vivo. Subsequent advances, both in terms of reagents and methods, have widened the scope of applications. However, the detection of low-affinity T cells is still lacking in many cases.

Here, we report the development of a novel, multivalent ‘spheromer’ system built on the scaffold of a self-assembling maxi-ferritin nanoparticle. As shown, the system has been engineered to be compatible with current pMHC (both MHC-I and MHC-II molecules) and SAv reagents that allows ease-of-use. The optimized spheromer assembly pipeline resulted in a very consistent reagent across multiple batches of synthesis with a relative ease of production, unlike the dodecamer. The defined geometry of the scaffold facilitated precise site-directed conjugation of pMHC, leading to a relatively homogenous reagent as assessed using a size-exclusion column. The spheromer bound cognate TCRs with a significantly higher avidity when compared to the tetramer, for both MHC-I (>50-fold) and MHC-II (>20-fold) molecules. Also, the low background contributed to the better signal to noise ratio observed in comparison to other pMHC-formulations tested. The improved TCR binding properties of the spheromer may in part also be due to better 2D binding kinetics owing to its larger diameter. This may provide a better surrogate than either the tetramer or dextramer for membrane embedded pMHC molecules that engage TCRs in vivo. This increased avidity and specificity enabled the detection of more potentially disease relevant, low-affinity T cells. Using the HLA-A*02:01 restricted influenza-M1 and HCMV-pp65 epitopes, we demonstrated that a significantly higher frequency of antigen-specific CD8⁺ T cells with a much more diverse TCR repertoire could indeed be detected with the spheromer. These results demonstrate that our engineered scaffold can be readily adapted with currently available reagents without a time-consuming systemic overhaul.

We further applied the spheromer technology to delineate the CD8⁺ T cell response to SARS-CoV-2 using a panel of peptides derived from multiple proteins (ORF1ab, S, M and N) that were validated for HLA-A*02:01 binding. Studies have shown that a T cell response can indeed be generated against multiple SARS-CoV-2 proteins. We observed a higher frequency of T cells against a few epitopes in the ORF1ab (P5, P10, P12, and P13) and S (P17 and P18) proteins in naïve, unexposed individuals. The high sequence similarity of these epitopes to hCoVs and the predominant memory phenotype of these T cells suggests that exposure to seasonal coronaviruses could contribute to the expansion of potentially cross-reactive T cells. Importantly, the frequency of T cells against a subset of these cross-reactive peptides (P5, P10, P12 and P17) was significantly higher in COVID-19 patients with mild symptoms. In contrast, T cells to unique ORF1ab derived peptides (P1 and P8) were higher in severely ill COVID-19 patients. These peptides (P1 and P8) have low sequence similarity to hCoVs. Overall, our data indicate that mild and severe COVID-19 patients elicit distinct T cell responses to particular SARS-CoV-2 epitopes. Also, the preferential recruitment of memory CD8⁺ T cells to cross-reactive epitopes likely contributes to their mild symptoms. These cross-reactive T cell responses may also contribute to the milder clinical symptoms observed in children when compared to adults since seasonal hCoVs infections are more frequent in children than adults. This study suggests that in addition to pre-existing cross-reactive memory CD4⁺ T cells reported previously, dissimilar SARS-CoV-2 epitope specific CD8⁺ T cell responses could also contribute to divergent COVID-19 clinical outcomes. The observation of CD8⁺ T cell responses to multiple SARS-CoV-2 proteins is consistent with previous studies. Accordingly, the data presented here suggests that the incorporation of additional non-spike epitopes into a vaccine could further bolster anti-viral T cell immunity.

Materials and Methods

Design, expression and characterization of multimeric protein scaffolds. In order to develop an optimized self-assembling protein scaffold for the multivalent presentation of peptide-MHC (pMHC) molecules, we designed and tested several (>30) protein constructs. All constructs were codon-optimized for expression in mammalian cells. Gene blocks (Integrated DNA Technologies) corresponding to individual constructs were cloned into a vector with a CMV/R promoter by gibson assembly (New England BioLabs) and sequence confirmed (Elim Biopharm). We first evaluated the heterologous recombinant expression of self-assembling proteins with different oligomeric states (n=12, 24 and 60). The sequences corresponding to mini-ferritin (12-mer, UniProt accession ID: POABT2), maxi-ferritin (24-mer, UniProt accession ID: Q8U2T8) and lumazine synthase (60-mer, UniProt accession ID: E6PLJ8) were cloned and expressed in Expi293F cells (Thermo Fisher Scientific) as per the manufacturer recommendations.

Briefly, 100 ml of Expi293F cells sub-cultured at a density of 3×10⁶ viable cells/ml in Expi293 expression medium (Thermo Fisher Scientific) were transfected with the expression plasmids complexed with ExpiFectamine 293 transfection reagent. Next day (˜18 h post-transfection), the cells were supplemented with a cocktail of enhancers. The cell cultures were further incubated for 4 d. Subsequently, the culture supernatants were harvested by centrifugation (2000 g, 30 mins, 4° C.) for protein purification. The supernatants were filtered (0.45 mm PES membrane filters, Thermo Fisher Scientific) and diluted with 20 mM Tris-HCl, pH 8. The proteins were bound to a HiTrap Q FF anion exchange column (Cytiva) using an AKTA pure 25 L1 system (Cytiva). An NaCl gradient (in 20 mM Tris-HCl, pH 8) was used to elute the bound proteins. The yield and purity of the multimeric protein scaffolds was estimated using a NuPAGE Bis-Tris 4-12% gradient gel system (Thermo Fisher Scientific). The homogeneity of the purified proteins was assessed using a Superdex 200 Increase 10/300 GL (Cytiva) size-exclusion column that was calibrated using a gel filtration standard (Bio-Rad).

On the basis of protein yield and homogeneity, we further optimized the maxi-ferritin scaffold for pMHC display by testing multiple linkers varying in length and rigidity. A list of all the evaluated linkers is given in FIG. 6A. Each construct was expressed and purified in mammalian cells as described above. The protein construct with linker (SG₂P)₂SG₂ (L6) was chosen for “spheromer” assembly based on yield and optimal radial projection from the scaffold. The sequence of the optimized maxi-ferritin scaffold is given in FIG. 6B. Site-directed functionalization (biotinylation) of the scaffold was performed using BirA biotin-protein ligase. The purified scaffold was incubated with components of the biotinylation reaction as per the manufacturer's recommendation (Avidity). The functionalized scaffold was subsequently separated from the free biotin using a Superdex 200 Increase 10/300 GL (Cytiva) size-exclusion column.

Next, the efficiency of protein biotinylation was assessed using a streptavidin gel-shift assay. Briefly, the protein was boiled at 90° C. for 7 mins before incubation on ice for 10 mins. Subsequently, a 2-fold molar excess of streptavidin (SAv, Agilent) was added to the protein and incubated further for an additional 10 mins on ice. The shift in mobility of the scaffold resulting from SAv binding was evaluated using the NuPAGE Bis-Tris 4-12% gradient gel system (Thermo Fisher Scientific).

Spheromer assembly and characterization. The spheromer assembly is a two-step process: i) Generation of a semi-saturated SAv-pMHC₂ complex, and ii) Conjugation of SAv-pMHC₂ to the functionalized maxi-ferritin scaffold. We optimized the reaction conditions for getting the maximum yield of SAv-pMHC₂ by varying the reactant concentrations, incubation time, agitation conditions and reaction temperature. We evaluated the formation of SAv-pMHC₂ by size-exclusion chromatography (Cytiva) and NuPAGE Bis-Tris 4-12% gradient gel system (Thermo Fisher Scientific). Subsequently, the spheromer assembly was generated by incubating SAv-pMHC₂ with the functionalized scaffold for 1 h at room temperature with mild rotation. We determined the stoichiometry of pMHC saturation on the spheromer by incubating the functionalized scaffold with increasing concentrations of pMHC and analyzing the resulting complex on a Superdex 200 Increase 10/300 GL (Cytiva) size-exclusion column.

We further purified the spheromer assembly using a size-exclusion column to mitigate the confounding effects from any unreacted SAv-pMHC₂. We also validated the conjugation of pMHC onto the functionalized scaffold by negative stain electron microscopy. 5 ml of the purified samples (0.005-0.5 mg/ml) was applied on glow discharged carbon-coated grids, blotted and stained with 1% uranyl formate according to standard protocols. Negative stained grids were imaged on an FEI Morgagni at 100 kV.

The number of pMHC molecules conjugated to the engineered maxi-ferritin scaffold was also quantified by ELISA using standard curves generated for pMHC and SAv. Briefly, test samples were coated on 96-well Nunc plates (Thermo Fisher Scientific) at 2 mg/ml in 50 ml PBS, pH 7.4 at 37° C. for 1 h. Plates were then washed with PBS containing 0.05% Tween-20 (PBST) and blocked with 3% skim milk in PBST for 1 h. The plates were washed and incubated at room temperature with 50 ml of HRP-conjugated anti-streptavidin IgG (Abcam) in blocking buffer at a predetermined dilution (1:5000) for 1 h for the detection of SAv. Alternatively, MHC-I and MHC-II molecules were detected using HRP-conjugated anti-HLA A2 IgG (LSBio) or HRP-conjugated anti-HLA DR IgG (LSBio). Plates were washed with PBST and developed with 75 ml/well of the substrate 3,3′,5,5′-tetramethylbenzidine (TMB) solution (MilliporeSigma). The reaction was stopped with 100 ml/well of ELISA stop solution for TMB (Thermo Fisher Scientific). The optical density at 450 nm was measured using the FlexStation 3 Multi-Mode Microplate Reader (Molecular Devices) and corrected for any non-specific background signal from ovalbumin coated wells.

Cloning, expression and purification of soluble TCRs. The soluble TCRs were expressed and purified as described previously. Briefly, for each TCR, the extracellular domains corresponding to the TCRα and TCRβ chains were codon-optimized for expression in insect cells and cloned independently into a baculovirus expression vector optimized for TCR expression by gibson assembly (New England BioLabs). The sequence confirmed (Elim Biopharm) plasmids were amplified in E. coli (New England BioLabs). Each plasmid was co-transfected with BestBac Linearized Baculovirus DNA (Expression Systems) into SF9 insect cells using Cellfectin II for the production of baculoviruses. The P1 stocks of TCRα and TCRβ baculoviruses of a given TCRαβ pair were titrated to ensure a 1:1 TCRαβ hetero-dimer formation and then co-transduced into Hi5 cells. After 3 d, the supernatant was collected by centrifugation. A precipitation mix (50 mM Tris-HCl (pH 8), 1 mM NiCl₂, and 5 mM CaCl₂) was added to the supernatant while stirring for 15 min at 25° C. The precipitation was subsequently removed by centrifugation and the supernatant was incubated with buffer-equilibrated Ni-NTA beads (Qiagen) for 4 h at 25° C. under mild mixing conditions. Then, the Ni-NTA beads were collected and washed with 20 mM imidazole in HBS (pH 7.2). The bound protein was eluted using 200 mM imidazole in HBS (pH 7.2). The TCRαβ heterodimer was further purified by a size-exclusion column (Superdex 200 Increase 10/300 GL (Cytiva)) using an AKTA pure 25 L1 system (Cytiva). The eluted fractions were pooled and analyzed for purity using SDS-PAGE.

MHCI protein purification and peptide exchange. In order to generate HLA-A*02:01 (MHCI) monomers, the corresponding α-chain and β2m protein constructs were over-expressed separately in E. coli. The protein was refolded from the inclusion bodies in the presence of a UV-cleavable peptide and biotinylated for downstream applications as described previously. After purification, the protein was concentrated and stored with 20% glycerol at −80° C. For each epitope specificity tested in this study, peptide exchange reactions were set up in a volume of 100 ml containing 0.2 mM peptide and 100 mg/ml HLA-A2*02:01 protein in PBS (pH 7.4). The reaction mixture was exposed to 365 nm UV-light irradiation for 20 mins using a Stratagene UV Stratalinker 2400 in 96-well U-shaped-bottom microplates (Corning). The plate was then transferred to 4° C. overnight to complete the exchange. The protein was subsequently buffer exchanged against PBS (pH 7.4) using Microcon centrifugal filters (10 kDa cut-off, MilliporeSigma) to remove the excess free peptide and subsequently spun at 13000 g for 15 mins at 4° C. to remove aggregates. The protein was filtered and stored at 4° C. until further use.

Purification of MHCII heterodimers and peptide exchange. The ectodomains of HLA-DRA, HLA-DRB1*04:01 and HLA-DRB1*15:01 were cloned into a CMV/R promoter-based vector by gibson assembly (New England BioLabs). The gene constructs were codon-optimized for mammalian expression. The sequence confirmed (Elim Biopharm) plasmids were amplified in E. coli. Plasmids encoding the α- and β-chains of a given MHCαβ hetero-dimer were co-transfected into Expi293F cells (Thermo Fisher Scientific) following the manufacturer recommendations. The transfected cells were enhanced ˜18-20 h post-transfection with the ExpiFectamine 293 transfection enhancers 1 and 2 (Thermo Fisher Scientific). The supernatant was harvested 5 d post-transfection and incubated with buffer-equilibrated Ni-NTA beads (Qiagen) for 5 h at 4° C. The Ni-NTA beads were then collected, washed (20 mM imidazole in HBS (pH 7.2)) and the bound protein was eluted under gravity flow with 200 mM imidazole in HBS (pH 7.2). The protein was buffer-exchanged to remove the imidazole and biotinylated using the BirA biotin-protein ligase reaction kit (Avidity) as per the manufacturer recommendations. The MHCII heterodimer was subsequently purified by via size-exclusion chromatography (Superdex 200 Increase 10/300 GL (Cytiva)) using an AKTA pure 25 L1 system (Cytiva). The eluted fractions were pooled and analyzed for purity and biotinylation efficiency using SDS-PAGE. Thrombin (Novagen) was used to cleave the invariant CLIP peptide from the purified MHCII molecules to enable exchange with the test peptide. After 2 h incubation of MHCII molecules with thrombin at room temperature, the reaction was stopped by the addition of a protease inhibitor cocktail (MilliporeSigma). The cleaved MHCII protein was incubated at 30° C. overnight in an aqueous solution of 1% octyl β-D-glucopyranoside, 0.1M NaCl, 50 mM citrate (pH 5.2), 1 mM EDTA, and 20 mg/mL test peptide for completion of exchange. Next day, the reaction was neutralized with 1M Tris-HCl (pH 8). The excess peptide was removed during buffer exchange against PBS (pH 7.4) using Microcon centrifugal filters (10 kDa cut-off, MilliporeSigma). The protein was further spun at 13000 g for 15 mins at 4° C. to remove aggregates and filtered before storing at 4° C. until further use.

Generation of pMHC multimer reagents. Here, we generated different multivalent formulations of a given pMHC specificity to enable comparative analysis. In order to ascribe the observed differences to the multimerization scaffold, all the multivalent pMHC formulations (tetramer, dextramer and spheromer) were made using the same stock of purified MHC molecules. The pMHC-tetramers were generated as described previously. Briefly, fluorophore-conjugated streptavidin (Invitrogen) was added to each pMHC monomer incrementally to achieve a 4:1 (pMHC:SAv) molar ratio. Next, streptavidin agarose was added to each tetramer for quenching any unbound, biotinylated pMHC. After filtration, biotinylated agarose beads were added to remove any unsaturated streptavidin molecules. The protein was filtered and stored at 4° C. until further use. We also used a previously described protocol for generating the pMHC-dextramers. The biotinylated pMHC molecules were incubated with fluorophore-conjugated streptavidin (Invitrogen) at a molar ratio of ˜3.5:1 (pMHC:SAv) for 30 mins at room temperature. To this mixture, biotin-dextran (MW=70 kDa, Thermo Fisher Scientific) was added at a molar ratio of ˜30:1 (pMHC:Dextran) and incubated further for another 30 mins at room temperature. The spheromer assembly has already been described above.

Binding affinity measurements using biolayer interferometry (BLI). Binding affinity for the cognate TCR-pMHC pairs was determined by BLI using an Octet QK instrument (ForteBio). The purified, soluble TCRs were captured onto amine reactive second-generation (AR2G) biosensors using the amine reactive second-generation reagent kit. The ligand-bound biosensors were then dipped into a decreasing concentration series (20 mM followed by 2-fold dilutions) of the indicated analytes in PBST (PBS with 0.05% Tween-20) to determine the binding kinetics. A series of unliganded biosensors dipped into the analytes served as controls for referencing. In addition, signals from analyte binding to an irrelevant TCR was used for non-specific binding correction. The traces were processed using ForteBio Data Analysis Software.

Lentiviral transduction for generating T cell lines. The T cell lines were generated as described previously. Briefly, gene blocks (Integrated DNA Technologies) corresponding to the TCRα and TCRβ chains of a given TCRab pair were cloned into the EF1a-MCS-GFP-PGK-puro lentiviral vector. Each sequence confirmed (Elim Biopharm) lentiviral plasmid was separately co-transfected with the gag-pol and VSV-G envelope plasmids into Lenti-X 293T cells (Takara Bio) cultured in DMEM media (Thermo Fisher Scientific) supplemented with 10% FBS (R&D Systems) and 100 U/ml of penicillin-streptomycin using FuGene (Promega) transfection reagent. After 72 h, lentiviruses for both TCRα and TCRβ constructs were harvested by collecting the culture supernatant. TCR-deficient Jurkat cells (α⁻β⁻) were transduced with the viral supernatant. TCR and CD3 expression was assessed by flow cytometry after staining the cells with anti-TCR α/β (PE) and anti-CD3 (BV421) antibodies for 30 mins on ice. The cells were washed, resuspended in FACS buffer (PBS with 1% BSA and 2 mM EDTA) and acquired on a BD LSRII flow cytometer. The data was analyzed using FlowJo (v10) software. If TCR expression after lentiviral transduction was <80%, enrichment for TCR expression was performed using anti-TCR α/β (APC) antibody in conjunction with anti-APC microbeads (Miltenyi Biotec).

Binding of T cell lines with pMHC multimers. The binding of pMHC to T cell lines was monitored by flow cytometry. pMHC multimers with Ax647 conjugated streptavidin (Invitrogen) were generated as described above. Binding curves (MFI) were determined using a concentration series of the pMHC multimer reagents. The cells were stained with pMHC multimers (tetramer, dextramer and spheromer) for 1 h in FACS buffer. The pMHC multimer staining was done at 4° C. or 25° C. for MHCI and MHCII restricted T cell specificities respectively. The cells were washed and subsequently stained with anti-CD3 (BV421) antibody for 20 mins on ice. Then the cells were washed twice, resuspended in FACS buffer and acquired on a Attune N×T Flow Cytometer (Thermo Fisher Scientific). The data was analyzed using FlowJo (v10) software.

Human biological sample collection. Peripheral blood mononuclear cells (PBMCs) from healthy donors were obtained from the Stanford Blood Center according to our IRB approved protocol. All healthy donor samples used in the current study were confirmed to be HLA-A*02:01+ and were collected between April 2018-February 2019 before the SARS-CoV-2 pandemic. The EBV and CMV infection status for these donors was also determined by the Stanford Blood Center. The COVID-19 patient sample collection for this study was conducted at the Stanford Medical Center under an IRB approved protocol (Protocol Director, Nadeau). We obtained samples from all COVID-19+ adults who had a positive-test result for the SARS-CoV-2 virus from analysis of nasopharyngeal swab specimens obtained at any point from March 2020-June 2020. Stanford Health Care clinical laboratory developed internal testing capability with a reverse-transcriptase based polymerase-chain-reaction assay. All participants consented prior to enrolling in the study. We obtained clinical data from Stanford clinical data electronic medical record system as per consented participant permission. This database contains all the clinical data available on all inpatient and outpatient visits to Stanford facilities. The data obtained included patients' demographic details, vital signs, laboratory test results, medication administration data, historical and current medication lists, historical and current diagnoses, clinical notes, and radiological results. Participants were excluded if they were taking any experimental medications (i.e., those medications not approved by a regulatory agency for use in COVID-19). The severity of COVID-19 illness was defined based on the symptom score described by Chen et al.

PBMC staining and flow cytometry. PBMCs were thawed in a water bath set at 37° C. and the cells were immediately transferred to warm RPMI media (Thermo Fisher Scientific) supplemented with 10% FBS (R&D Systems) and 100 U/ml of penicillin-streptomycin. After washing, the cells were filtered (70 mm cell strainer) and rested for 1 h at 37° C. CD8⁺ T cells were enriched from PBMCs by negative selection using a FITC-conjugated antibody cocktail against non-CD8⁺ T cells (anti-CD14, anti-CD19, anti-CD33 and anti-γδ TCR) followed by magnetic bead depletion using anti-FITC microbeads (Miltenyi Biotec). The enriched CD8⁺ T cells were washed and resuspended in FACS buffer for staining. All pMHC-multimer (MHCI) staining were done for 1 h at 4° C. after incubating the cells with Human TruStain FcX (BioLegend) for 15 mins. In order to compare the frequency of viral (influenza and HCMV) antigen specific T cells detected using tetramer or spheromer, each sample was divided equally after CD8⁺ T cell enrichment and stained with M1-A*02:01 (Ax647) and pp65-A*02:01 (PE) formulated as tetramer or spheromer. Both the pMHC-multimer formulations were used at a monomeric concentration of 100 nM. The gag-A*02:01 (Ax488) pMHC-multimer (200 nM) was used as irrelevant ‘negative’ control. The cells were subsequently stained with anti-CD19 (BV510), anti-gd TCR (BV510), anti-CD33 (BV510), anti-CD3 (PE/Cyanine7), anti-CD8 (BUV396), anti-CD4 (BV786), anti-CCR7 (PE/Dazzle 594), anti-CD45RA (BV711) and an amine-reactive viability stain (Live/dead fixable aqua dead cell stain kit; Invitrogen) for 30 mins, washed, resuspended in FACS buffer and acquired on a BD LSRII flow cytometer. The data was analyzed using FlowJo (v10) software and antigen specific T cell enumerated as described previously.

For the simultaneous detection of multiple SARS-CoV-2 epitopes (described below) using the spheromer technology, we adapted a combinatorial staining approach developed previously. Briefly, each peptide was assigned a unique fluorophore-combination tag that allows the simultaneous detection of 2ⁿ-1 specificities in a sample, where n is the number of distinct fluorophore labels. The relative concentrations for pMHC monomers associated with each fluorophore label (Ax647, eFluor 450, PE and PE/Cyanine7) was experimentally determined. Four T cell lines with distinct antigen specificities (M1-A*02:01, pp65-A*02:01, BMLF1-A*02:01 and BHW58-A*02:01) were mixed at a pre-determined ratio with TCR-deficient Jurkat cells (α⁻β⁻) and stained with a pool of spheromers, wherein each cognate pMHC was associated with a unique fluorescent tag. The cells were further labeled with anti-CD3 (FITC) for 30 mins, washed, resuspended in flow cytometry buffer and acquired on a BD LSRII flow cytometer. The data was analyzed to determine the optimal concentration for pMHC monomers associated with each fluorophore label (Ax647; 100 nM, eFluor 450; 125 nM, PE; 75 nM and PE/Cyanine7; 50 nM) that generated maximum separation between the distinct T cell lines. The gag-A*02:01 pMHC-spheromer defined by the fluorophore-combination tag (Ax647+eFluor 450+PE+PE/Cyanine7) was used as irrelevant ‘negative’ control.

After staining PBMC samples with spheromer pools displaying SARS-CoV-2 epitopes, the cells were subsequently stained with anti-CD19 (BV510), anti-gd TCR (BV510), anti-CD33 (BV510), anti-CD3 (FITC), anti-CD8 (BUV396), anti-CD4 (BV786), anti-CCR7 (PE/Dazzle 594), anti-CD45RA (BV711) and an amine-reactive viability stain (Live/dead fixable aqua dead cell stain kit; Invitrogen) for 30 mins. ˜0.1×10⁶ cells from each COVID-19 patient sample was also separately stained (without spheromer pools) with anti-CD19 (BV510), anti-γδ TCR (BV510), anti-CD33 (BV510), anti-CD3 (FITC), anti-CD8 (BUV396), anti-CD4 (BV786), anti-CCR7 (PE/Dazzle 594), anti-CD45RA (BV711), anti-HLA A2 (Ax700) antibody and an amine-reactive viability stain (Live/dead fixable aqua dead cell stain kit; Invitrogen) for 30 mins on ice. The cells were washed, resuspended in FACS buffer and processed using a BD LSRII flow cytometer. The data was analyzed using FlowJo (v10) software. Selection of SARS-CoV-2 peptides and sequence conservation analysis The complete genome sequence for SARS-CoV-2 isolate SARS-CoV-2/USA/WA-CDC-WA1/2020 (GenBank accession ID: MN985325) was obtained from the NCBI database. The binding of all possible 9-mers from SARS-CoV-2 ORF1ab, S, M and N proteins to HLA-A*02:01 was predicted following the immune epitope database and analysis resource (IEDB) recommendations. The peptide binding predictions were cross validated using the SYFPEITHI algorithms. We further prioritized peptides based on the biochemical properties of amino acids at positions P2, P5 and P9.

The binding of selected peptides to HLA-A*02:01 was further experimentally validated by an MHC stabilization assay using the transporter associated with antigen processing (TAP) deficient T2 cell line expressing HLA-A*02:01. Briefly, T2 cells were incubated with 10 mM of the test peptide (GenScript) in AIM V serum free media (Thermo Fisher Scientific) for 1 h at 37° C. The cells were then transferred to a lower temperature (26° C.) for another 14 h, before returning them to 37° C. for 3 h prior to antibody staining. The cells were washed free of any unbound peptide and incubated with anti-HLA A2 (PE) antibody and an amine-reactive viability stain (Live/dead fixable aqua dead cell stain kit; Invitrogen) for 30 mins on ice. Subsequently, cells were washed, resuspended in FACS buffer and acquired on a BD LSRII flow cytometer. T2 cells incubated in AIM V serum free media alone (no peptide) served as a negative control. The list of SARS-CoV-2 peptides evaluated using the pheromer technology in this study are listed in Table S1.

To perform a sequence conservation analysis of the peptides selected from SARS-CoV-2 across other seasonal hCoVs, we obtained representative whole genome sequences for 229E (GenBank accession ID: MN306046), HKU1 (HCoV_HKU1/SC2628/2017, GenBank accession ID: KY983584), NL63 (HCoV_NL63/UF-2/2015, GenBank accession ID: KX179500) and OC43 (HCoV_OC43/Seattle/USA/SC9430/2018, GenBank accession ID: MN306053) from the NCBI database. The binding of all possible 9-mers from ORF1ab, S, M and N proteins to HLA-A*02:01 for each of the seasonal hCoV reference strains listed above was predicted following the immune epitope database and analysis resource (IEDB) recommendations.

We then filtered the peptides based on percentile rank (<5.0). A lower percentile rank indicates higher affinity. This was done to restrict the search for cross-reactive peptides in hCoVs that are potentially functional owing to their ability to bind HLA-A*02:01, a pre-requisite to activate T cells. We then calculated the pairwise sequence similarity score for each of the selected SARS-CoV-2 peptides against all filtered seasonal hCoV peptides using the sequence manipulation suite. The sequence similarity score was calculated allowing for amino acid substitutions (GA, VLI, FYW, ST, KR, DE and NQ) with similar biochemical properties. The list of seasonal hCoV peptides identified based on the similarity score is given in Table S1. The sequence similarity (%) and the percentile rank are also mentioned.

Single-cell paired αβ-TCR sequencing. Multiplexed αβ-TCR sequencing was done following previously established protocols. In brief, single spheromer-positive CD8⁺ T cells (for influenza-M1, HCMV-pp65 and SARS-CoV-2 specificities) were sorted into 96-well plates containing 12 ml OneStep RT-PCR buffer (Qiagen). Reverse transcription was done using the OneStep RT-PCR kit (Qiagen) and the resulting cDNA was used for TCRα and TCRβ amplification using multiplex primers. DNA barcodes were also incorporated within the amplified sequences before processing the samples in a single MiSeq2×300 bp sequencing run. The paired sequencing reads were joined, demultiplexed, and mapped to the human TCR reference dataset available at the international ImMunoGeneTics information system (IMGT) as reported previously.

CD8⁺ T cells detected using the spheromer by comparing them to tetramer or dextramer derived sequences retrieved from the VDJdb database. For each antigen specificity, we implemented the GLIPH2 algorithm to quantify the number of clusters (characterized by a distinct TCR CDR3b motif) that were unique to the spheromer or had an overlap with TCR sequences reported using the tetramer or dextramer. Briefly, the GLIPH2 algorithm compared the antigen specific TCRs (input dataset) against a reference dataset of 273,920 distinct TCR CDR3b sequences from 12 healthy individuals to generate clusters with unique TCR CDR3b motifs that are significantly enriched (p-value≤0.05, Fisher's exact test) in the input dataset as previously described (28). We also analyzed the SARS-CoV-2 epitope specific TCR sequences identified from unexposed, healthy individuals using the spheromer by implementing the GLIPH2 algorithm. The TCR sequences from COVID-19 patient samples for this analysis were obtained from a published dataset. The inclusion of multiple statistical measurements in the GLIPH2 output accounting for Vb gene usage biases, CDR3b length distribution (relevant only for local motifs), cluster size, HLA allele usage, and clonal expansion facilitates the calling of high-confidence specificity groups.

In vitro stimulation of T cell lines. The stimulation assay was done as previously described. The assay was setup in 96-well clear round bottom microplates (Corning) with a volume of 200 ml during all incubation penicillin-streptomycin and pulsed with 100 mM of the test peptide for 3 h at 37° C. The cells were washed and co-cultured with Jurkat cells expressing an exogenous TCR of interest (100,000 cells/well) in RPMI media (Thermo Fisher Scientific) supplemented with 10% FBS (R&D Systems) and 100 U/ml of penicillin-streptomycin for 16 h. Next day, the cells were washed with FACS buffer and stained with anti-CD3 (APC) and anti-CD69 (PE) antibodies for 20 min at 4° C. Cells were washed, resuspended in FACS buffer and analyzed on an Attune N×T Flow Cytometer (Thermo Fisher Scientific). The data was analyzed using FlowJo (v10) software.

Statistical analysis. R statistical package was used to perform the Fisher's exact test to compute TRBV gene enrichment across different pMHC formulations using the fisher.test function. Dimensionality reduction analysis were also performed in R. UMAPs to visualize multiparametric flow cytometry data were generated using the “umap” package. Principal component analysis to evaluate the divergent response to SARS-CoV-2 epitopes between mild and severe COVID-19 patients was done using “FactoMineR” and “factoextra” packages. Additional data and statistical analyses were done in GraphPad Prism. The statistical details for each experiment are also provided in the legends.

### Example 2

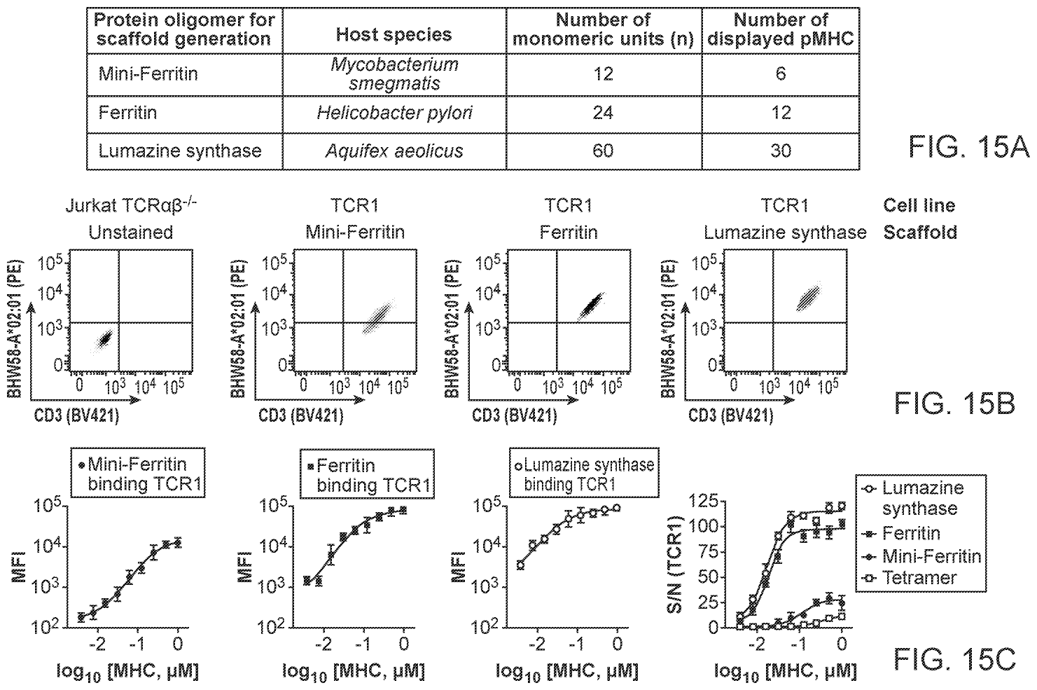

As shown in Example 1, an engineered maxi-ferritin from Pyrococcus furiosus can act as a scaffold to increase the avidity of interaction between a peptide-MHC (pMHC) and its cognate T cell receptor (TCR). As shown in FIG. 15, other self-assembling polypeptides are useful for this purpose, including other ferritin polypeptides. FIG. 15 provides a list of oligomeric proteins that have been characterized for the display of pMHC, and provides data for the staining of a cognate T cell line (TCR1) with the indicated pMHC-multimer formulations; and quantification of BHW58-A*02:01 binding to different pMHC-multimer formulations measured by flow cytometry (mean±SD).

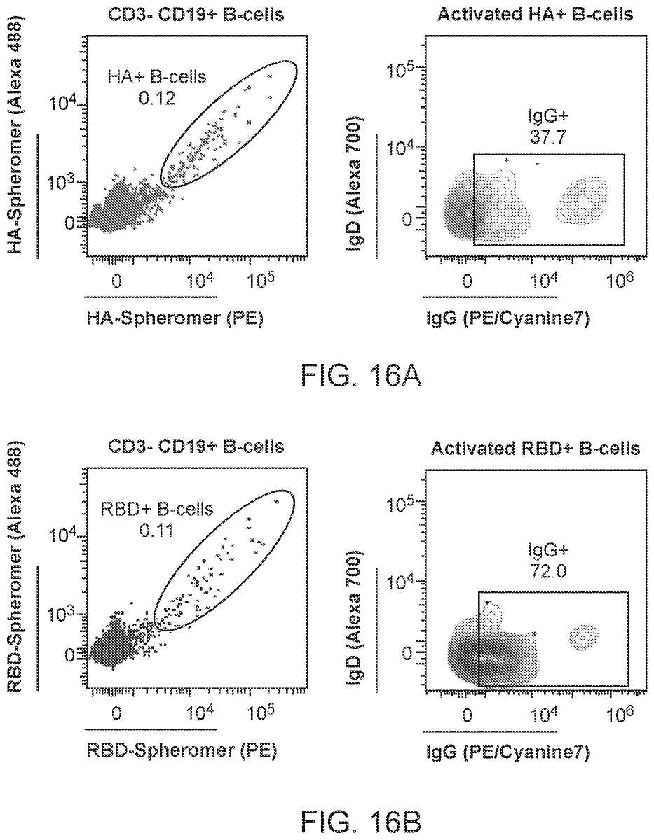

The spheromer scaffold staining was shown in Example 1 to be effective in binding to T cells. The staining is shown to FIG. 16 to be effective in staining B cells. PBMCs isolated from volunteers ˜3-4 weeks after (A) influenza or (B) SARS-CoV-2 vaccination were stained with (A) influenza hemagglutinin (HA) or (B) SARS-CoV-2 receptor binding domain (RBD) displayed on the spheromer scaffold.

Table 1 lists HLA-A*02:01 restricted SARS-CoV-2 peptides. The subset of these peptides that are conserved across human coronaviruses are associated with mild symptoms in COVID-19 patients, e.g. the peptides of SEQ ID NO:-81. These conserved peptide sequences are useful in vaccine design to aid in protection against SARS-CoV-2 infection.

TABLE 1

| | | SARS-CoV-2 | | | |
|---|---|---|---|---|---|
| | | | | | Percentile |
| HLA-allele | Protein | Start | Sequence | SEQ ID | rank¹ |
| HLA-A*02:01 | ORF1ab | 84 | VMVELVAEL | SEQ ID NO: 1 | 0.50 |
| | | 103 | TLGVLVPHV | SEQ ID NO: 2 | 1.40 |
| | | 1675 | YLATALLTL | SEQ ID NO: 3 | 0.30 |
| | | 3013 | SLPGVFCGV | SEQ ID NO: 4 | 0.70 |
| | | 3467 | VLAWLYAAV | SEQ ID NO: 5 | 0.50 |
| | | 3482 | FLNRFTTTL | SEQ ID NO: 6 | 0.60 |
| | | 3710 | TLMNVLTLV | SEQ ID NO: 7 | 0.20 |
| | | 3732 | SMWALIISV | SEQ ID NO: 8 | 0.20 |
| | | 3871 | VLLSVLQQL | SEQ ID NO: 9 | 1.20 |
| | | 4032 | MLFTMLRKL | SEQ ID NO: 10 | 1.30 |
| | | 4094 | ALWEIQQVV | SEQ ID NO: 11 | 0.30 |
| | | 4515 | TMADLVYAL | SEQ ID NO: 12 | 0.30 |
| | | 4725 | IFVDGVPFV | SEQ ID NO: 13 | 0.28 |
| | Spike | 269 | YLQPRTFLL | SEQ ID NO: 14 | 0.30 |
| | | 417 | KIADYNYKL | SEQ ID NO: 15 | 0.70 |
| | | 691 | SIIAYTMSL | SEQ ID NO: 16 | 1.40 |
| | | 976 | VLNDILSRL | SEQ ID NO: 17 | 1.10 |
| | | 983 | RLDKVEAEV | SEQ ID NO: 18 | 0.90 |
| | | 1220 | FIAGLIAIV | SEQ ID NO: 19 | 0.40 |
| | Membrane | 15 | KLLEQWNLV | SEQ ID NO: 20 | 0.40 |
| | | 26 | FLFLTWICL | SEQ ID NO: 21 | 0.40 |
| | | 61 | TLACFVLAA | SEQ ID NO: 22 | 1.50 |
| | | 89 | GLMWLSYFI | SEQ ID NO: 23 | 0.20 |
| | Nucleoprotein | 222 | LLLDRLNQL | SEQ ID NO: 24 | 0.80 |
| | | 316 | GMSRIGMEV | SEQ ID NO: 25 | 1.80 |
| HLA-A*01:01 | ORF1ab | 221 | FIDTKRGVY | SEQ ID NO: 26 | 0.06 |
| | | 241 | YTERSEKSY | SEQ ID NO: 27 | 0.01 |
| | | 1321 | PTDNYITTY | SEQ ID NO: 28 | 0.01 |
| | | 1415 | VVDYGARFY | SEQ ID NO: 29 | 0.02 |
| | | 1891 | EIDPKLDNY | SEQ ID NO: 30 | 0.02 |
| | | 3437 | GTDLEGNFY | SEQ ID NO: 31 | 0.01 |
| | | 4083 | TCDGTTFTY | SEQ ID NO: 32 | 0.02 |
| | | 4163 | CTDDNALAY | SEQ ID NO: 33 | 0.01 |
| | | 4198 | KSDGTGTIY | SEQ ID NO: 34 | 0.01 |
| | | 4842 | ISDYDYYRY | SEQ ID NO: 35 | 0.01 |
| | | 4867 | VVDKYFDCY | SEQ ID NO: 36 | 0.02 |
| | | 5130 | DTDFVNEFY | SEQ ID NO: 37 | 0.01 |
| | | 6268 | QADVEWKFY | SEQ ID NO: 38 | 0.05 |
| | | 6669 | AMDEFIERY | SEQ ID NO: 39 | 0.02 |
| | Spike | 136 | CNDPFLGVY | SEQ ID NO: 40 | 0.27 |
| | | 152 | WMESEFRVY | SEQ ID NO: 41 | 0.26 |
| | | 258 | WTAGAAAYY | SEQ ID NO: 42 | 0.06 |
| | | 604 | TSNQVAVLY | SEQ ID NO: 43 | 0.03 |
| | | 733 | KTSVDCTMY | SEQ ID NO: 44 | 0.12 |
| | | 865 | LTDEMIAQY | SEQ ID NO: 45 | 0.01 |
| | Nucleoprotein | 78 | SSPDDQIGY | SEQ ID NO: 46 | 0.10 |
| | | 104 | LSPRWYFYY | SEQ ID NO: 47 | 0.29 |
| | Membrane | 39 | YANRNRFLY | SEQ ID NO: 48 | 0.10 |
| | | 171 | ATSRTLSYY | SEQ ID NO: 49 | 0.03 |
| | | 188 | AGDSGFAAY | SEQ ID NO: 50 | 0.10 |
| | | 213 | SSDNIALLV | SEQ ID NO: 51 | 0.14 |
| HLA-B*40:01 | ORF1ab | 56 | VEKGVLPQL | SEQ ID NO: 52 | 0.06 |
| | | 376 | SEVGPEHSL | SEQ ID NO: 53 | 0.01 |
| | | 385 | AEYHNESGL | SEQ ID NO: 54 | 0.04 |
| | | 389 | NESGLKTIL | SEQ ID NO: 55 | 0.05 |
| | | 725 | EETGLLMPL | SEQ ID NO: 56 | 0.20 |
| | | 744 | GETLPTEVL | SEQ ID NO: 57 | 0.01 |
| | | 1141 | HEVLLAPLL | SEQ ID NO: 58 | 0.03 |
| | | 1442 | NETLVTMPL | SEQ ID NO: 59 | 0.19 |
| | | 1548 | GEVITFDNL | SEQ ID NO: 60 | 0.07 |
| | | 1613 | HEGKTFYVL | SEQ ID NO: 61 | 0.04 |
| | | 1705 | GEAANFCAL | SEQ ID NO: 62 | 0.07 |
| | | 2069 | TEVVGDIIL | SEQ ID NO: 63 | 0.06 |
| | | 2087 | TEEVGHTDL | SEQ ID NO: 64 | 0.16 |
| | | 2325 | AEWFLAYIL | SEQ ID NO: 65 | 0.10 |
| | | 2618 | AELAKNVSL | SEQ ID NO: 66 | 0.01 |
| | | 4474 | HEETIYNLL | SEQ ID NO: 67 | 0.04 |
| | | 4645 | AESHVDTDL | SEQ ID NO: 68 | 0.06 |
| | | 5267 | QEYADVFHL | SEQ ID NO: 69 | 0.01 |
| | | 5479 | REVLSDREL | SEQ ID NO: 70 | 0.03 |
| | | 6970 | TEHSWNADL | SEQ ID NO: 71 | 0.13 |
| | Spike | 168 | FEYVSQPFL | SEQ ID NO: 72 | 0.06 |
| | | 339 | GEVFNATRF | SEQ ID NO: 73 | 0.16 |
| | | 464 | FERDISTEI | SEQ ID NO: 74 | 0.09 |
| | | 989 | AEVQIDRLI | SEQ ID NO: 75 | 0.13 |
| | | 1016 | AEIRASANL | SEQ ID NO: 76 | 0.01 |
| | | 1257 | DEDDSEPVL | SEQ ID NO: 77 | 0.22 |
| | Nucleoprotein | 159 | LQLPQGTTL | SEQ ID NO: 78 | 0.47 |
| | | 322 | MEVTPSGTW | SEQ ID NO: 79 | 0.73 |
| | | 215 | GDAALALLL | SEQ ID NO: 80 | 0.86 |
| | Membrane | 136 | SELVIGAVI | SEQ ID NO: 81 | 0.14 |

SARS-CoV-2 peptide panel. Spheromers displaying these peptides in the context of HLA as listed were used to assess the CD8⁺ T cell response in unexposed individuals and COVID-19 patients.

### REFERENCES AND NOTES

C. Huang et al., Clinical features of patients infected with 2019 novel coronavirus in Wuhan, China. Lancet 395, 497-506 (2020).
F. Krammer, SARS-CoV-2 vaccines in development. Nature 586, 516-527 (2020).
K. H. D. Crawford et al., Dynamics of neutralizing antibody titers in the months after SARS-CoV-2 infection. J Infect Dis, (2020).
M. Hellerstein, What are the roles of antibodies versus a durable, high quality T-cell response in protective immunity against SARS-CoV-2? Vaccine X 6, 100076 (2020).
J. Seow et al., Longitudinal evaluation and decline of antibody responses in SARS-CoV-2 infection. medRxiv, 2020.2007.2009.20148429 (2020).
A. Wajnberg et al., Robust neutralizing antibodies to SARS-CoV-2 infection persist for months. Science, (2020).
A. P. Ferretti et al., Unbiased Screens Show CD8(+) T Cells of COVID-19 Patients Recognize Shared Epitopes in SARS-CoV-2 that Largely Reside outside the Spike Protein. Immunity, (2020).
A. Grifoni et al., Targets of T Cell Responses to SARS-CoV-2 Coronavirus in Humans with COVID-19 Disease and Unexposed Individuals. Cell 181, 1489-1501 e1415 (2020).
N. Le Bert et al., SARS-CoV-2-specific T cell immunity in cases of COVID-19 and SARS, and uninfected controls. Nature 584, 457-462 (2020).
J. Mateus et al., Selective and cross-reactive SARS-CoV-2 T cell epitopes in unexposed humans. Science 370, 89-94 (2020).
A. Nelde et al., SARS-CoV-2-derived peptides define heterologous and COVID-19-induced T cell recognition. Nat Immunol, (2020).
Y. Peng et al., Broad and strong memory CD4 (+) and CD8 (+) T cells induced by SARS-CoV-2 in UK convalescent COVID-19 patients. bioRxiv, (2020).
L. C. Wu, D. S. Tuot, D. S. Lyons, K. C. Garcia, M. M. Davis, Two-step binding mechanism for T-cell receptor recognition of peptide MHC. Nature 418, 552-556 (2002).
M. M. Davis et al., Ligand recognition by alpha beta T cell receptors. Annu Rev Immunol 16, 523-544 (1998).
J. D. Altman et al., Phenotypic analysis of antigen-specific T lymphocytes. Science 274, 94-96 (1996).
P. Batard et al., Dextramers: new generation of fluorescent MHC class 1/peptide multimers for visualization of antigen-specific CD8+ T cells. J Immunol Methods 310, 136-148 (2006).
G. Dolton et al., More tricks with tetramers: a practical guide to staining T cells with peptide-MHC multimers. Immunology 146, 11-22 (2015).
J. Huang et al., Detection, phenotyping, and quantification of antigen-specific T cells using a peptide-MHC dodecamer. Proc Natl Acad Sci USA 113, E1890-1897 (2016).
G. Dolton et al., Comparison of peptide-major histocompatibility complex tetramers and dextramers for the identification of antigen-specific T cells. Clin Exp Immunol 177, 47-63 (2014).
C. Rius et al., Peptide-MHC Class I Tetramers Can Fail To Detect Relevant Functional T Cell Clonotypes and Underestimate Antigen-Reactive T Cell Populations. J Immunol 200, 2263-2279 (2018).
L. E. Bevers, E. C. Theil, Maxi- and mini-ferritins: minerals and protein nanocages. Prog Mol Subcell Biol 52, 29-47 (2011).
G. Ueda et al., Tailored design of protein nanoparticle scaffolds for multivalent presentation of viral glycoprotein antigens. Elife 9, (2020).
A. Razvi, J. M. Scholtz, Lessons in stability from thermophilic proteins. Protein Sci 15, 1569-1578 (2006).
P. J. Schatz, Use of peptide libraries to map the substrate specificity of a peptide-modifying enzyme: a 13 residue consensus peptide specifies biotinylation in Escherichia coli. Biotechnology (N Y) 11, 1138-1143 (1993).
J. S. Klein, S. Jiang, R. P. Galimidi, J. R. Keeffe, P. J. Bjorkman, Design and characterization of structured protein linkers with differing flexibilities. Protein Eng Des Sel 27, 325-330 (2014).
D. V. Bagaev et al., VDJdb in 2019: database extension, new analysis infrastructure and a T-cell receptor motif compendium. Nucleic Acids Res 48, D1057-D1062 (2020).
A. Han, J. Glanville, L. Hansmann, M. M. Davis, Linking T-cell receptor sequence to functional phenotype at the single-cell level. Nat Biotechnol 32, 684-692 (2014).
H. Huang, C. Wang, F. Rubelt, T. J. Scriba, M. M. Davis, Analyzing the Mycobacterium tuberculosis immune response by T-cell receptor clustering with GLIPH2 and genome-wide antigen screening. Nat Biotechnol 38, 1194-1202 (2020).
W. S. DeWitt, 3rd et al., Human T cell receptor occurrence patterns encode immune history, genetic background, and receptor specificity. Elife 7, (2018).
I. Miconnet et al., Large TCR diversity of virus-specific CD8 T cells provides the mechanistic basis for massive TCR renewal after antigen exposure. J Immunol 186, 7039-7049 (2011).
L. F. Su, B. A. Kidd, A. Han, J. J. Kotzin, M. M. Davis, Virus-specific CD4(+) memory-phenotype T cells are abundant in unexposed adults. Immunity 38, 373-383 (2013).
W. Yu et al., Clonal Deletion Prunes but Does Not Eliminate Self-Specific alphabeta CD8(+) T Lymphocytes. Immunity 42, 929-941 (2015).
W. W. Kwok et al., Frequency of epitope-specific naive CD4(+) T cells correlates with immunodominance in the human memory repertoire. J Immunol 188, 2537-2544 (2012).
J. J. Moon et al., Naive CD4(+) T cell frequency varies for different epitopes and predicts repertoire diversity and response magnitude. Immunity 27, 203-213 (2007).
J. J. Obar, K. M. Khanna, L. Lefrancois, Endogenous naive CD8+ T cell precursor frequency regulates primary and memory responses to infection. Immunity 28, 859-869 (2008).
Y. Kim et al., Immune epitope database analysis resource. Nucleic Acids Res 40, W525-530 (2012).
H. Rammensee, J. Bachmann, N. P. Emmerich, O. A. Bachor, S. Stevanovic, SYFPEITHI: database for MHC ligands and peptide motifs. Immunogenetics 50, 213-219 (1999).
E. W. Newell, L. O. Klein, W. Yu, M. M. Davis, Simultaneous detection of many T-cell specificities using combinatorial tetramer staining. Nat Methods 6, 497-499 (2009).
D. M. Altmann, R. J. Boyton, SARS-CoV-2 T cell immunity: Specificity, function, durability, and role in protection. Sci Immunol 5, (2020).
M. M. Davis, T cell analysis in vaccination. Curr Opin Immunol 65, 70-73 (2020).
J. J. Melenhorst et al., Detection of low avidity CD8(+) T cell populations with coreceptor-enhanced peptide-major histocompatibility complex class I tetramers. J Immunol Methods 338, 31-39 (2008).
H. Reijonen, W. W. Kwok, Use of HLA class II tetramers in tracking antigen-specific T cells and mapping T-cell epitopes. Methods 29, 282-288 (2003).
S. K. Saini et al., Empty peptide-receptive MHC class I molecules for efficient detection of antigen-specific T cells. Sci Immunol 4, (2019).
T. J. Scriba et al., Ultrasensitive detection and phenotyping of CD4+ T cells with optimized HLA class II tetramer staining. J Immunol 175, 6334-6343 (2005).
P. Serra et al., Increased yields and biological potency of knob-into-hole-based soluble MHC class II molecules. Nat Commun 10, 4917 (2019).
P. Zimmermann, N. Curtis, Coronavirus Infections in Children Including COVID-19: An Overview of the Epidemiology, Clinical Features, Diagnosis, Treatment and Prevention Options in Children. Pediatr Infect Dis J 39, 355-368 (2020).
N. Principi, S. Bosis, S. Esposito, Effects of coronavirus infections in children. Emerg Infect Dis 16, 183-188 (2010).

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

That which is claimed is:

1. A spheromer MHC-peptide complex, comprising:
a scaffold comprising at least 12 self-assembling polypeptide monomers, conjugated to a plurality of peptide major histocompatibility (MHC) complexes.

2. The spheromer of claim 1, wherein the self-assembling polypeptide is a ferritin polypeptide.

3. The spheromer of claim 2, wherein the ferritin is a maxi-ferritin and the scaffold comprises 24 monomers.

4. The spheromer of claim 3, wherein the maxi-ferritin is Pyrococcus furiosus maxi-ferritin, or Helicobacter pylori maxi-ferritin.

5. The spheromer of claim 2, wherein the self-assembling polypeptide is a mini-ferritin and the scaffold comprises 12 monomers.

6. The spheromer of claim 5, wherein the mini-ferritin is Mycobacterium smegmatis mini-ferritin.

7. The spheromer of claim 1, wherein the self-assembling polypeptide is a lumazine synthase polypeptide and the scaffold comprises 60 monomers.

8. The spheromer of claim 7, wherein the lumazine synthase is Aquifex aeolicus lumazine synthase.

9. The spheromer of any of claims 1-8, wherein the self-assembling scaffold monomers are conjugated to the peptide MHC complexes by a biotin-streptavidin interaction.

10. The spheromer of any of claims 1-9, wherein the self-assembling scaffold monomers are genetically modified to comprise a biotinylation signal sequence.

11. The spheromer of any of claims 1-8, wherein the self-assembling scaffold monomers are conjugated to the peptide MHC complexes by sortase-tag, where the pMHCs are modified to include the -LPXTG sortase A tag while the scaffold is modified to include an oligo glycine (G)n, with n=3-7 at the terminus to allow protein-protein ligation when incubated with the Sortase A ligase; or Spy-tag, where the SpyCatcher sequence is appended to the C-terminus of the pMHC, while the scaffold is modified to include the Spy-tag, and the modified proteins are irreversibly conjugated upon incubation

12. The spheromer of claim 10 or claim 11, wherein the scaffold monomer modification is separated from the self-assembling polypeptide sequence by a peptide linker.

13. The spheromer of any of claims 1-12, comprising a detectable label.

14. A spheromer composition comprising one or more SARS-CoV-2 peptides.

15. The spheromer composition of claim 14, wherein the Sars-CoV-2 peptide is one or more of SEQ ID NO:1-81; and may comprise each of SEQ ID NO:1-81.

16. A method of detecting, activating or separating an immune cell according to specificity of it's antigen receptor, the method comprising:
contacting a population of the immune cells with a spheromer according to any of claims 1-15.

17. The method of claim 16, wherein the immune cells are T cells.

18. The method of claim 17, wherein the immune cells are B cells.

19. The method of any of claims 16-18, wherein the immune cells comprise a mixed population of B and T cells.
